SEQUENCE LISTING (9 sequences)

SEQ ID NO: 1 - protein, 60 aa, Homo sapiens
AMVRELLIQFYKSTRFKPTRIIFYRDGVSEGQFQQVLHHELLAIREACIKLEKDYQPGIT

SEQ ID NO: 2 - protein, 60 aa, Mus musculus; identical to SEQ ID NO: 1

SEQ ID NO: 3 - protein, 58 aa, Melanogaster
SITLEHLRVYKEYRNAYPDHIIYYRDGVSDGQFPKIKNEELRCIKQACDKVGCKPKIC

SEQ ID NO: 4 - protein, 60 aa, Caenorhabditis elegans
YMVRELLVQFYRNTRFKPARIVVYRDGVSEGQFFNVLQYELRAIREACMMLERGYQPGIT

SEQ ID NO: 5 - protein, 60 aa, Schizosaccharomyces pombe
DIVVYLLQGFRAMTKQQPQRIIYFRDGTSEGQFLSVINDELSQIKEACHSLSPKYNPKIL

SEQ ID NO: 6 - protein, 60 aa, Homo sapiens
IEDLSYMVRELLIQFYKSTRFKPTRIIFYRDGVPEGQLPQILHYELLAIRDACIKLEKDY

SEQ ID NO: 7 - protein, 60 aa, Homo sapiens
IQDLAAMVRELLIQFYKSTRFKPTRIIFYRDGVSEGQFQQVLHHELLAIREACIKLEKDY

SEQ ID NO: 8 - protein, 60 aa, Homo sapiens
IQDLASMVRELLIQFYKSTRFKPTRIIFYRDGVSEGQFRQVLYYELLAIREACISLEKDY

SEQ ID NO: 9 - protein, 60 aa, Homo sapiens
IQDLTNMVRELLIQFYKSTRFKPTRIIYYRGGVSEGQMKQVAWPELIAIRKACISLEEDY

(19) United States
(12) Patent Application Publication
(10) Pub. No.: US 2022/0184143 A1
(43) Pub. Date: Jun. 16, 2022

(54) COMPOSITIONS AND METHODS OF MODULATING CASOTRANSMITTER SIGNALING

(71) Applicant: CASE WESTERN RESERVE UNIVERSITY, Cleveland, OH (US)

(72) Inventor: Jonathan S. Stamler, Cleveland, OH (US)

(21) Appl. No.: 17/432,225

(22) PCT Filed: Feb. 19, 2020
(86) PCT No.: PCT/US2020/018842

§ 371 Date: Aug. 19, 2021

Related U.S. Application Data
(60) Provisional application No. 62/807,477, filed on Feb. 19, 2019.

(51) Int. Cl. A61K 35/74; A61K 31/04; A61K 33/04

(52) U.S. Cl. CPC A61K 35/74; A61K 2035/115; A61K 33/04; A61K 31/04

(57) ABSTRACT

A method of treating a disorder associated gasotransmitter signaling in a subject in need thereof includes modulating the microbiota of the subject to modulate microbiota gasotransmitter production and modification of the subject's proteome to treat the disorder in the subject.

### RELATED APPLICATION

This application claims priority from U.S. Provisional Application No. 62/807,477, filed Feb. 19, 2019 the subject matter of which IS incorporated herein by reference in its entirety.

### GOVERNMENT FUNDING

This invention was made with government support under Grant Nos. R01GM099921, T32GM007250, F30AG054237, and R35HL135789 awarded by The National Institutes of Health. The United States government has certain rights to the invention.

### BACKGROUND

Nitric oxide (NO), hydrogen sulfide (H₂S), and carbon monoxide (CO) are gasotransmitters (gaseous signaling molecules) with many different functions in human biology, including specific roles in cardiovascular physiology. NO is used by the endothelium to signal surrounding smooth muscle in the walls of arterioles to relax, resulting in vasodilation and increased blood flow to hypoxic tissues. NO is also involved in regulating smooth muscle proliferation, platelet function, and neurotransmission, and plays a role in host defense and cellular injury/stress. NO also has general cellular signaling and metabolic functions and is broadly implicated in disease pathogenesis and mammalian health, including longevity and age-related diseases. H₂S has multiple functions in physiology including regulation of neurological function, intestinal inflammation, ischemia reperfusion injury, vasomotor tone regulation, and angiogenesis. CO has been found to play a key beneficial role in various inflammatory and cardiovascular diseases, and in settings associated with oxidative stress broadly. Among the various inflammatory related disorders, inflammatory bowel disease (IBD), lung inflammation, psoriasis, mid-ear infection-induced inflammation, uveitis, and burn- and injury-related inflammation can be effectively treated by CO.

These gaseous mediators are synthesized through enzymatic and non-enzymatic pathways. NO production classically occurs in an enzymatic manner via nitric oxide synthase isoenzymes, with enzymatic or non-enzymatic reduction of nitrite/nitrate serving as an alternative pathway. H₂S is produced by three principal enzymes (cystathionine γ-lyase, cystathionine β-synthase, and 3-mercaptopyruvate transferase), with non-enzymatic generation via glutathione and other sulfane sulfur redox pathways. The biological effects of NO and H₂S occur predominately through small molecule interactions and post-translational protein thiol modifications.

CO is produced through the activity of heme oxygenases (HO-1 and HO-2). These enzymes regulate the catabolism of heme and play an important role in the modulation of a variety of responses, such as stress response and circadian rhythm. Studies have shown that CO has anti-inflammatory, anti-proliferative, and anti-apoptotic effects when the concentrations of CO in carrier gas (air) ranges from 10 to 250 ppm.

Currently, there is a great need in the art for diagnostics, prophylaxis, ameliorations, and treatments for medical conditions relating to NO, H₂S, and CO synthesis and/or NO bioactivity.

### SUMMARY

Embodiments described herein relate to compositions and methods of modulating microbiota gasotransmitter production and particularly relates to compositions and methods of modulating the microbiota of a subject to modulate microbiota gasotransmitter production (e.g., increase or decrease production), modify the subject's proteome, and treat disorders associated gasotransmitter signaling, such as disorders associated with NO/SNO deficiency. It was found that microbiota-dependent modification of host proteomes by gasotransmitters, such as NO, H₂S, and CO, represent general mechanisms by which resident microbiota of a host can control or regulate host function. Modulation of the resident microbiota by administration of compositions that increase or decrease microbiota gasotransmitter (e.g., NO, H₂S, and CO) production and/or levels in the host can be used to treat disorders associated with gasotransmitter deficiency or excess.

In some embodiments, the compositions and methods described herein can promote microbiota NO, H₂S, and/or CO production in vivo in a subject and treat/attenuate conditions of NO, H₂S, and/or CO deficiency or insufficiency in a subject, particularly in the gastrointestinal tract (e.g., oral cavity, stomach, and/or intestine) of a subject, by, for example, fostering growth and/or colonization of microbes in the gastrointestinal tract of the subject.

In other embodiments, the compositions and methods described herein can inhibit microbiota NO, H₂S, and/or CO production in vivo in a subject and treat/attenuate conditions of NO, H₂S, and/or CO excess in a subject, particularly in the gastrointestinal tract (e.g., oral cavity, stomach, and/or intestine) of a subject, by, for example, inhibiting growth and/or colonization of microbes in the gastrointestinal tract of the subject.

In some embodiments, the compositions and methods can include one or more isolated bacteria capable of enhancing and/or producing NO, H₂S, and/or CO in the microbiota of the subject. The bacteria can include commensal bacteria of the gastrointestinal tract. The commensal bacteria can include bacteria of the genera Alistipes, Akkermansia, Anaerofilum, Bacteroides, Blautia, Bifidobacterium, Butyrivibrio, Clostridium, Coprococcus, Dialister, Dorea, Fusobacterium, Eubacterium, Faecalibacterium, Lachnospira, Lactobacillus, Odoribacter, Oscillospira, Parabacteroides, Phascolarctobacterium, Peptococcus, Peptostreptococcus, Prevotella, Roseburia, Ruminococcus, Streptococcus, or Subdoligranulum.

In some embodiments, the composition can include microbes (bacteria, fungi, yeast) capable of producing nitric oxide and optionally a substrate of nitric oxide synthase and/or nitrate reductase (or cytochrome oxidase in case of yeast). The nitric oxide synthase and/or nitrate reductase (or cytochrome oxidase in case of yeast) can be a native enzyme expressed by the bacteria or be a recombinant enzyme expressed or overexpressed by genetically modified bacteria. The optional substrate of nitric oxide synthase or nitrate reductase can include at least one of arginine, a nitrate, a nitrite, or a salt thereof. The bacteria and the substrate of nitric oxide synthase and/or nitrate reductase can be administered at the same time or in separate compositions.

In some embodiments, the nitrate can be an organic nitrate, for example, from a botanical source. The botanical source of nitrate can include one or more of beet root, kale, artichoke, holy basil, gymnema sylvestre, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof.

In some embodiments, the bacteria and optionally the substrate are administered to the subject at an amount effective to increase S-nitrosylation of proteins in the subject. The bacteria and optionally the substrate can also be administered to the subject at an amount effective to increase SNO levels in blood or tissue of the subject.

In some embodiments, the bacteria and microbes, and optionally the substrate of nitrate, can be administered to improve gut (or skin/vaginal) health of the subject by increasing local gasotranmitter production to increase S-nitrosylation of local tissues and in other mcirobes, thereby regulating their functions.

In other embodiments, the bacteria/microbes, and optionally the substrate, can be administered to the subject at an amount effective to treat at least one of acute coronary syndrome, acute lung injury (ALI), acute myocardial infarction (AMI), acute respiratory distress syndrome (ARDS), pulmonary fibrosis, asthma, chronic obstructive pulmonary disorder (COPD), arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, irritable bowel syndrome, constipation, anal fissures, anal spasm, duodenal and gastric ulcers, pyloric stenosis, sphincter of oddi constriction, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasia, ischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, neurodegenerative disease (e.g., Alzheimer's disease, Huntington's disease, AIDs dementia, or Parkinson's disease), peripheral arterial disease (PAD), peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, or wounds to tissues or organs.

Other embodiments described herein relate to a method of treating a disorder associated with a change in gene expression in a subject in need thereof. The method includes modulating the microbiota of the subject to modulate microbiota gasotransmitter production and modify the subject's proteome to treat the disorder in the subject.

Still other embodiments relate to a method of treating a disorder associated with change in microRNA activity and/or gene expression in a subject in need thereof. The method includes modulating the microbiota of the subject to modulate microbiota gasotransmitter production and modify the subject's proteome to treat the disorder in the subject.

Other embodiments relate to a method of treating a disorder associated with gasotransmitter signaling in a subject in need thereof. The method includes modulating the microbiota of the subject to modulate microbiota gasotransmitter production and modify the subject's proteome to treat the disorder in the subject.

In some embodiments, the microbiota and/or subject can be administered inhibitors of gasotransmitter production by the microbiota at an amount effective to decrease S-nitrosylation of proteins in the subject.

In other embodiments, the microbiota and/or subject can be modulated to increase S-nitrosylation of proteins in the subject.

In other embodiments, the microbiota and/or subject can be administered inhibitors of gasotransmitter production by microbiota at an amount effective to decrease S-sulfhydration of proteins in the subject.

In other embodiments, the microbiota and/or subject can be modulated to increase S-sulfhydration of proteins in the subject.

In some embodiments, the microbiota and/or subject can be administered inhibitors of gasotransmitter production by microbiome at an amount effective to decrease CO microbiota production in the subject.

In other embodiments, the microbiota and/or subject can be modulated to increase CO microbiota production in the subject.

### BRIEF DESCRIPTION OF THE DRAWINGS

FIGS. 1(A-M) illustrate microbiota-derived NO mediates widespread protein S-nitrosylation in C. elegans, including Argonaute proteins. (A) Robust S-nitrosylation of the C. elegans proteome by B. subtilis. Silver stain of endogenous SNO-proteins (following SNO-RAC) was performed on lysates harvested from C. elegans grown either on wild type B. subtilis 1A1 (WT) or Δnos B. subtilis (Δnos), treated with (SNO-proteome; left panel) or without (Control; middle panel) ascorbate (Asc). Coomassie blue stain of total proteome loading controls (right panel). Gels are representative of three experiments. (B) Quantification of gels in A (n=3, ±SEM). *, differs from WT by ANOVA with Dunnett's test (p<0.05). (C) S-nitrosylation of the C. elegans proteome by E. coli. Silver stain of endogenous SNO-proteins (following SNO-RAC) was performed on lysates harvested from C. elegans grown either on wild type E. coli (WT) or ΔnarG E. coli (ΔnarG), and either treated with (SNO-proteome; left panel) or without (Control; middle panel) ascorbate. Coomassie blue stain of total proteome loading controls (right panel). Gels are representative of three experiments. (D) Quantification of gels in C (n=3, ±SEM). *, differs from WT by ANOVA with Dunnett's test (p<0.05). (E) S-nitrosylation of C. elegans ALG-1 by NO derived from B. subtilis NOS. ALG-1 immunoblot following SNO-RAC (+Asc) from lysates as in A. -Asc serves as a control. Total ALG-1 loading control is also shown. Gels are representative of three experiments. (F) Quantification of gels in E (n=3, ±SEM). *, differs from WT by ANOVA with Dunnett's test (p<0.05). (G) S-nitrosylation of C. elegans ALG-1 by NO derived from E. coli nitrate reductase NarG. ALG-1 immunoblot following SNO-RAC (+Asc) from lysates as in A. Total ALG-1 loading control is also shown. Gels are representative of three experiments. (H) Quantification of gels in G (n=3, ±SEM). *, differs from WT by ANOVA with Dunnett's test (p<0.05). (I) Robust S-nitrosylation of the C. elegans proteome by small amounts of B. subtilis: effect of titration. Coomassie blue stain of endogenous SNO-proteins (following SNO-RAC) was performed on lysates harvested from C. elegans grown either on wild type (WT) B. subtilis 1A1 (100%) or Δnos (Δnos) B. subtilis 1A1 (0%) or a mixture comprising 10% WT and 90% Δnos; the SNO-proteome, -Asc control and total proteome loading controls are shown (left to right, respectively). Gels are representative of three experiments. (J) Quantification of gels in I (n=3, ±SEM). *, differs from WT by ANOVA with Dunnett's test (p<0.05). (K) S-nitrosylation of C. elegans ALG-1 by small amounts of B. subtilis NOS. Immunoblot of endogenous ALG-1 (following SNO-RAC) was performed on lysates harvested from C. elegans grown on wild type (WT) B. subtilis 1A1 (100%), Δnos (Δnos) B. subtilis 1A1 (0%), or mixtures comprising 10% WT and 90% Δnos, 25% WT and 75% Δnos, 50% WT and 50% Δnos, and 75% WT and 25% Δnos. A lower exposure autoradiography film is shown (middle). Total ALG-1 loading control is also shown. Gels are representative of three experiments. (L) Quantification of gels in J (n=3, ±SEM). *, differs from WT by ANOVA with Dunnett's test (p<0.05). (M) Bacterial colony forming units (CFU) per C. elegans from worms cultured with either WT B. subtilis (WT) or Δnos B. subtilis (Δnos).

FIGS. 2(A-G) illustrates AGO2 Cys691 is a primary locus of S-nitrosylation. (A) Endogenous S-nitrosylation of human AGO2. Immunoblot for AGO2 in HEK293 cells following SNO-RAC±ascorbate control (Asc). AGO2 loading control is shown. Gels are representative of three experiments. (B) Quantification of gels in A (n=3, ±SEM). A.U., arbitrary units. *, differs from +Asc by ANOVA with Dunnett's test (p<0.05). (C) S-nitrosylation of AGO2 by exogenous NO. Immunoblot for AGO2 in HEK293 cells following SNO-RAC±NO donor (CysNO). Total AGO2 loading control is shown. Gels are representative of three experiments. (D) Quantification of gels in C (n=3, ±SEM). *, differs from -CysNO by ANOVA with Dunnett's test (p<0.05). (E) Locus of S-nitrosylation in AGO2. Peptides containing the Cys691 site of S-nitrosylation identified by LC-MS/MS from 4 independent experiments. SNO-Cys were labelled with iodoacetamide using switch methodology (Jaffrey and Snyder, 2001). Conditions are as in C. (F) Validation of AGO2-Cys691 S-nitrosylation by site-directed mutagenesis. Immunoblot for SNO-AGO2 in HEK293 cells transfected with either WT-AGO2 (WT) or Cys691 mutant AGO2 (C691S) after treatment with CysNO, as in C. Total AGO2 loading control is shown. Gels are representative of three experiments. (G) Quantification of gels in F (n=3, ±SEM). *, differs from WT by ANOVA with Dunnett's test (p<0.05). See also FIG. 6.

FIGS. 3(A-D) illustrate S-nitrosylation of Argonaute proteins at a phylogenetically-conserved Cys. (A) Site of S-nitrosylation in human AGO2 is conserved in C. elegans ALG-1. Amino acid sequence alignment of H. sapiens AGO2 (SEQ ID NO: 1) and its orthologs (SEQ ID Nos: 2-9) from different eukaryotic species (top), and alignment of human AGO1-4 homologs (bottom). Human AGO2 SNO-site Cys691 is conserved in eukaryotes. (B) ALG-1-C855S mutant C. elegans is refractory to endogenous S-nitrosylation. Immunoblot for SNO-ALG-1 (immunoblot for ALG-1 following SNO-RAC) in lysates from WT or ALG-1-C855S C. elegans. Total ALG-1 loading control is shown. Gel is representative of three experiments. (C) Quantification of data in B (n=3, ±SEM). *, differs from WT ALG-1 nematodes by ANOVA with Dunnett's test (p<0.05). (D) 3D crystal structure of AGO1 in complex with GW182 hook motif (orange) showing the conserved SNO-site cysteine 689 (analogous to AGO2 Cys691) in red and the miRNA-mRNA complex in yellow.

FIGS. 4(A-M) illustrate conserved cysteine nitrosylation site in Argonaute proteins mediates an essential interaction with GW182 proteins. (A-D) Nitric oxide inhibits the interaction between GW182 and AGO2. (A) Immunoblot for GW182 following immunoprecipitation of AGO2 in the absence or presence of the NO donor DETA-NO (NO). HEK293 cells were transfected with FLAG-AGO2 and immunoprecipitation was carried out with αFLAG antibody. Gels are representative of three experiments. (B) Quantification of gels in A (n=3, ±SEM). *, differs from —NO by ANOVA with Dunnett's test (p<0.05). (C) FLAG immunoblot, following immunoprecipitation of endogenous GW182 with GW182 antibody. Conditions are as in A. Gels are representative of three experiments. (D) Quantification of gels in C (n=3, ±SEM). *, differs from -NO (-DETA-NO) by ANOVA with Dunnett's test (p<0.05). (E-H) Cys 691 in AGO2 is required for interaction with GW182. (E) GW182 immunoblot following immunoprecipitation of AGO2. HEK293 cells were transfected with either Myc-AGO2 (WT) or Myc-C691S mutant AGO2 (C691S). Immunoprecipitation was performed with antibody against Myc. (F) Quantification of gels in E (n=3, ±SEM). *, differs from WT-AGO2 by ANOVA with Dunnett's test (p<0.05). (G) FLAG immunoblot following immunoprecipitation of GW182 as in C. HEK293 cells were transiently transfected with either FLAG-WT-AGO2 or FLAG-AGO2-C691S. Gels are representative of three experiments. (H) Quantification of gels in G (n=3, ±SEM). *, differs from WT-AGO2 by ANOVA with Dunnett's test (p<0.05). (I) AlN-1 immunoblot from either let-7(n2853) WT ALG-1 or C855S-ALG-1 animals following IP with ALG-1 antibody, using lysates from animals co-cultured on WT B. subtilis (WT) or Δnos B. subtilis (Δnos). Total AIN-1 input is shown. (J) Quantification of gels in I (n=3, ±SEM). *, differs from WT B. subtilis by ANOVA with Dunnett's test (p<0.05). (K-M) Inhibition by NO of AGO2 activity mediated through S-nitrosylation of Cys691. (K) miRNA activity assays in HeLa cells using a luciferase reporter containing seven let-7 miRNA binding sites upon co-transfection with AGO2 WT in the absence or presence of DETA-NO (NO). Values presented are luciferase readings normalized for GFP. *, differs from —NO by ANOVA with Dunnett's test (p<0.05). (L) miRNA activity reporter assays as in K upon co-transfection of the AGO2-C691S mutant in either the absence or presence of NO. (M) miRNA activity reporter assays as in K and L, upon co-transfection with either AGO2 WT or AGO2 C691S mutant in the absence of NO (n=3, ±SEM). *, differs from WT by ANOVA with Dunnett's test (p<0.05). See also FIGS. 6-7.

FIGS. 5(A-C) illustrate microbe initiated S-nitrosylation of ALG-1 influences C. elegans developmental timing via microRNA activity. (A) B. subtilis-derived NO inhibits miRNA activity through modification of Cys855 in ALG-1. qPCR analysis of lin-41 repression in let-7(n2853) worms at late developmental stages (L3/L4). Values are presented relative to lin-41 mRNA levels at their respective early developmental stages (L1/L2), which have been normalized to 1. *, differs from their respective lin-41 mRNA levels at L1/L2 stage by ANOVA with Dunnett's test (p<0.05). Data is representative of three independent experiments (n=3, ±SEM). (B) miRNA mediated regulation of developmental timing is dependent on microbiota-derived NO modification of Cys855 in ALG-1. Vulval bursting scored by plotting percent worm death in let-7(n2853) animals. N=number of worms. *, differs from let-7(n2853) incubated with B. subtilis (B. sub) (p=0.003) by Chi square test with Bonferroni correction. (C) Model depicts microbiome mediated regulation of host development via S-nitrosylation of Argonaute proteins. nos, Nitric oxide synthase; narG, Nitrate reductase.

FIGS. 6(A-B) illustrate S-nitrosylation of AGO2 by endogenous eNOS is enhanced by the NO donor DETA-NO in HEK293 cells. Related to FIG. 2. (A) Immunoblot with □AGO2 antibody following SNO-RAC (+Asc) on lysates prepared from either untreated or DETA-NO (500 μM) treated cells. -Asc (control for SNO-RAC) and total AGO2 controls are also shown. (B) Immunoblot with NOS3 (eNOS) antibody using increasing quantities of HEK293 cell lysates. Actin is shown as the loading control.

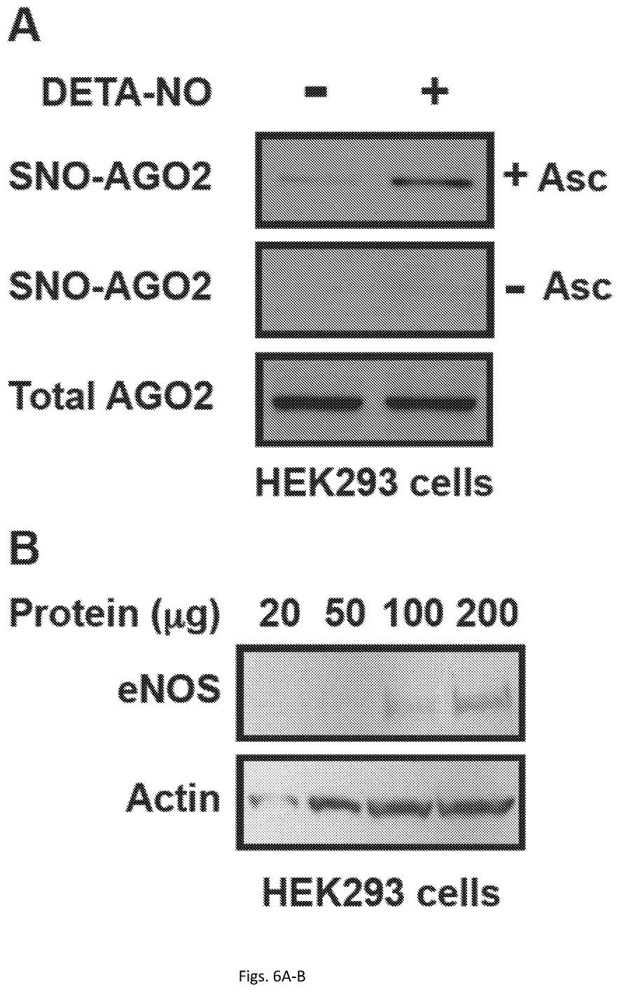

FIGS. 7(A-B) illustrate the effects of S-nitrosylation and SNO-site mutation on AGO2 activity and interactions. Related to FIG. 4. (A) Mutation of AGO2 Cys691 has little effect on AGO2 interaction with microRNA or target mRNA in cultured cells. Relative levels of AGO2-associated miR210 and EFNA3 mRNA, measured by qPCR from HEK 293 cells that had been transfected either with FLAG-tagged WT-AGO2 (WT) or FLAG-tagged C691S-AGO2 (C691S), after immunoprecipitation of AGO2 with αFLAG antibody. *, differs from WT by ANOVA with Dunnett's test (p<0.05). (B) Nitric oxide inhibits the interaction between endogenous AGO2 and GW182. Immunoprecipitation of endogenous AGO2 and GW182 are shown in the left and right panels respectively, along with controls NO treatment.

FIG. 8 illustrates mutation of AGO2 C691 does not prevent AGO2-dependent silencing activity. Related to FIG. 4. Comparable levels of β-arrestin2 knockdown are observed with β-arrestin2 specific siRNA (relative to non-specific siRNA control) in HEK 293 cells transfected with either WT FLAG-AGO2 or FLAG-AGO2-C691S. WT FLAG-AGO2 and FLAG-AGO2-C691S expression are shown; GAPDH loading control. *, differs from non-specific siRNA control by ANOVA with Dunnett's test (p<0.05).

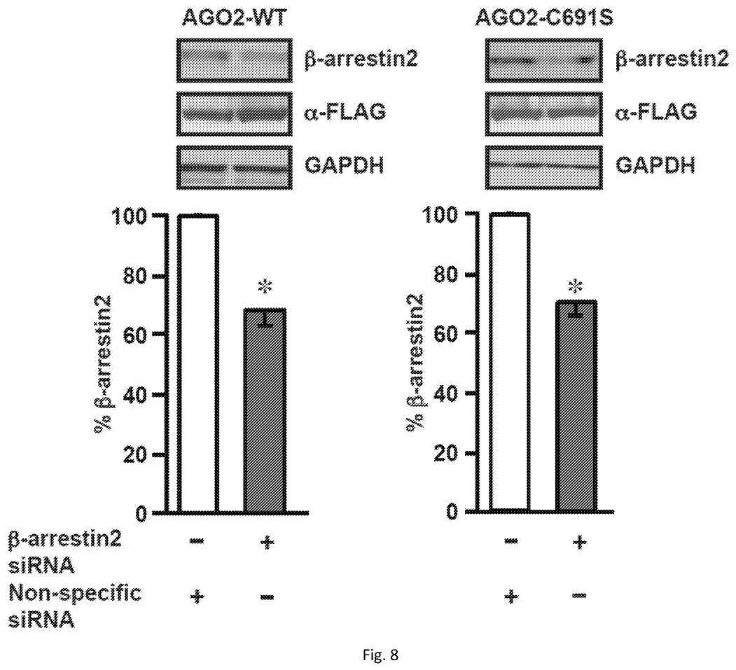

FIGS. 9(A-C) illustrate levels of lin-41 mRNA and let-7 miRNA at late larval stages (L3/L4) are similar in C. elegans N2 and let-7(n2853) strains cultured on WT B. subtilis (WT) vs. nos B. subtilis. Related to FIG. 5. (A) Relative levels of lin-41 mRNA, as determined by qPCR, in C. elegans N2 worms at early (L1/L2) and late (L3/L4) developmental stages. Values are presented relative to lin-41 at L1/L2, which has been normalized to 1. *, differs from early larval stages (L1/L2) by ANOVA with Dunnett's test (p<0.05). (B) qPCR analysis of let-7 miRNA expression in C. elegans N2 worms at early (L1/L2) and late developmental stages (L3/L4). Values are presented relative to let-7 (on WT B. subtilis diet) at L1/L2, which has been normalized to 1. (C) qPCR analysis of let-7 microRNA expression in C. elegans let-7(n2853) at early (L1/L2) and late (L3/L4) developmental stages at 21° C. Values are presented relative to let-7 (on WT B. subtilis diet) at L1/L2, which has been normalized to 1.

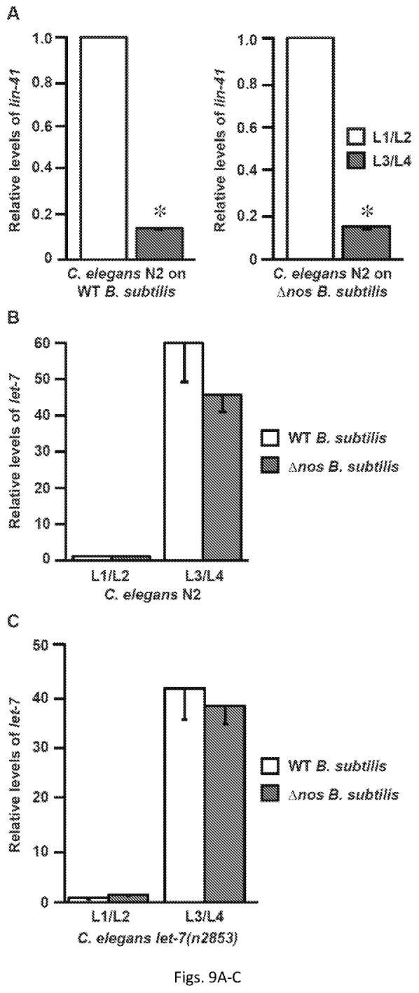

FIGS. 10(A-B) illustrate E. coli nitrate reductase mediates S-nitrosylation of C. elegans DAF-16. Related to FIG. 5. (A) Robust S-nitrosylation of C. elegans DAF-16 by bacterial nitrate reductase NarG. DAF-16 immunoblot following SNO-RAC from worm lysates. Total DAF-16 loading control is also shown. Gels are representative of three experiments. (B) Quantification of gels in A (n=3, ±SEM). *, differs from WT by ANOVA with Dunnett's test (p<0.05).

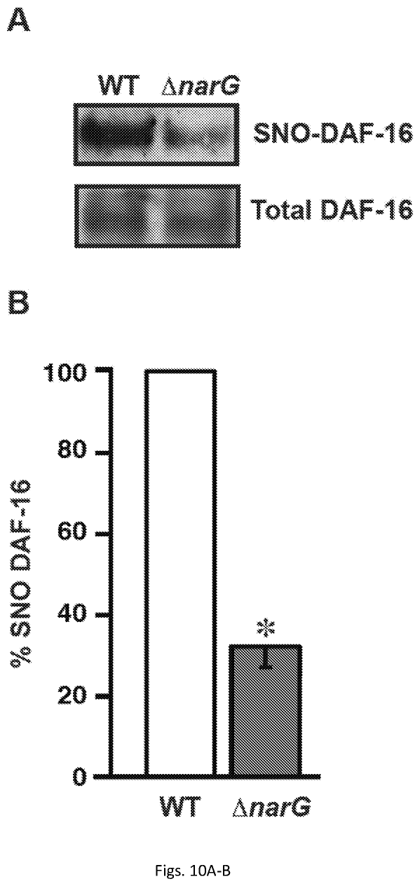

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The term “abundance” as it relates to a microbial taxa refers to the presence of one microbial taxa as compared to another microbial taxa in a defined microbial niche, such as the gastrointestinal (GI) tract, or in the entire host organism (e.g., a human or a laboratory animal model of disease).

The “colonization” of a host organism refers to the non-transitory residence of a bacterium or other microbial organism in a niche.

“Diversity of a microbial community” or “microbial diversity” as used herein refers to the diversity found in the microbiota within a given niche or host subject. Diversity can relate to the number of distinct microbial taxa and/or richness of the microbial taxa within the niche or host and can be expressed, e.g., using the Shannon Diversity index (Shannon entropy), alpha-beta diversity, total number of observed OTUs, or Chaol index, as described herein.

The term “dysbiosis of the gastrointestinal microbiota” refers to an imbalanced state of the microbiota of the GI tract (e.g., in the stomach, small intestine, or large intestine).

The term “microbiome” refers to the genetic content of the communities of microbes that live in and on a subject (e.g., a human subject), both sustainably and transiently, including eukaryotes, archaea, bacteria, and viruses (including bacterial viruses (e.g., phage)), wherein “genetic content” includes genomic DNA, RNA such as ribosomal RNA and messenger RNA, the epigenome, plasmids, and all other types of genetic information. In some embodiments, microbiome specifically refers to genetic content of the communities of microorganisms in a niche.

The term “microbiota” refers to the community of microorganisms that occur (sustainably or transiently) in and on a subject (e.g., a human subject), including eukaryotes, archaea, bacteria, yeast, fungi, and viruses (including bacterial viruses, e.g., phage). In some embodiments, microbiota specifically refers to the microbial community in a niche.

The terms “modulate the microbiota” or “modulating the microbiota” refer to changing the state of the microbiota. Changing the state of the microbiota may include changing the structure and/or function of the microbiota. A change in the structure of the microbiota is, e.g., a change in the relative composition of a taxa, e.g., in one or more regions of the GI tract such as the cecum, ascending colon, transverse colon, descending colon, sigmoid colon, and/or rectum. In an embodiment, a change in the structure of the microbiota comprises a change in the abundance of a taxa, e.g., relative to another taxa or relative to what would be observed in the absence of the modulation. Modulation of the microbiota may also include a change in a function of the microbiota, such as a change in microbiota gene expression, level of a gene product (e.g., RNA or protein), and/or metabolic output of the microbiota. Functions of the microbiota may also include pathogen protection, nutrition, host metabolism, and immune modulation. Modulation of the structure or function of the microbiota may additionally induce a change in one or more functional pathway of the host (e.g., a change in gene expression, level of a gene product, and/or metabolic output of a host cell or host process) as a result of a change in the microbiota structure or its function.

The term “engineered bacterial cell” or “engineered bacteria” refers to a bacterial cell or bacteria that have been genetically modified from their native state. For instance, an engineered bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Engineered bacterial cells disclosed herein may comprise exogenous nucleotide sequences on plasmids. Alternatively, engineered bacterial cells may comprise exogenous or heterologous nucleotide sequences stably incorporated into their chromosome.

The term, a “heterologous” gene or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell or genome.

The term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures.

The term a “non-native” nucleic acid sequence refers to a nucleic acid sequence not normally present in a bacterium, e.g., an extra copy of an endogenous sequence, or a heterologous sequence, such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence. The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered bacteria of the invention comprise a gene cassette that is operably linked to a directly or indirectly inducible promoter that is not associated with said gene cassette in nature.

The term “gut” refers to the organs, glands, tracts, and systems that are responsible for the transfer and digestion of food, absorption of nutrients, and excretion of waste. In humans, the gut comprises the gastrointestinal (GI) tract. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

The term “microorganism” refers to an organism or microbe of microscopic, submicroscopic, or ultramicroscopic size that typically consists of a single cell. Examples of microrganisms include bacteria, viruses, parasites, fungi, certain algae, yeast, and protozoa. In some aspects, the microorganism is engineered (“engineered microorganism”) to express one or more proteins or other compounds (synthetic or naturally occurring) and release them into its environment. In certain embodiments, the engineered microorganism is an engineered bacterium.

The term “non-pathogenic bacteria” refers to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (e.g., U.S. Pat. Nos. 6,835,376; 6,203,797; 5,589,168; 7,731,976). Naturally pathogenic bacteria may be genetically engineered to provide reduce or eliminate pathogenicity.

The term “probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (e.g., U.S. Pat. Nos. 5,589,168; 6,203,797; 6,835,376). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

Embodiments described herein relate to compositions and methods of modulating microbiota gasotransmitter production and particularly relates to compositions and methods of modulating the microbiota of a subject to modulate microbiota gasotransmitter production (e.g., increase or decrease production), modify the subject's proteome, and treat disorders associated gasotransmitter signaling, such as disorders associated with NO/SNO deficiency and/or disorders benefiting from gasotransmitter increases (e.g., anal fissure/spasm). It was found that microbiota-dependent modification of host proteomes by gasotransmitters, such as NO, H₂S, and CO, represent a general mechanism by which resident microbiota of a host can control host function. Modulation of the resident microbiota by administration of compositions that increase or decreases microbiota gasotransmitter (e.g., NO, H₂S, and CO) production and/or levels in the host can be used to treat disorders associated gasotransmitter deficiency or excess.

In some embodiments, the compositions and methods described herein can promote microbiota NO, H₂S, and/or CO production in vivo in a subject and treat/attenuate conditions of NO, H₂S, and/or CO deficiency or insufficiency in a subject, particularly in the gastrointestinal tract (e.g., oral cavity, stomach, and/or intestine) of a subject, by fostering growth and/or colonization of microbes in the gastrointestinal tract.

In some embodiments, the compositions and methods can include one or more isolated bacteria capable of enhancing and/or producing NO, H₂S, and/or CO in the microbiota of the subject. For example, the compositions can include one or more isolated bacteria capable of promoting nitric oxide synthesis or reducing nitrate to nitrite. Such isolated bacteria can express or overexpress nitric oxide synthase or nitrate reductase. In other embodiments, the isolated bacteria can express or overexpress cystathionine γ-lyase, cystathionine β-synthase, and 3-mercaptopyruvate transferase for H₂S production or heme oxygenases (HO-1 and HO-2) for CO production. The compositions may also include one or more pharmaceutically acceptable carriers or excipients.

In some embodiments, the compositions capable of enhancing and/or producing NO, H₂S, and/or CO in the microbiota can include probiotic compositions or preparations thereof, e.g., derived from bacterial cultures that are generally recognized as safe (GRAS) or known commensal or probiotic microbes. Examples of bacteria that can be used in probiotic compositions described herein can include, but are not limited to, organisms classified as genera Bacteroides, Blautia, Clostridium, Fusobacterium, Eubacterium, Ruminococcus, Peptococcus, Peptostreptococcus, Akkermansia, Faecalibacterium, Roseburia, Prevotella, Bifidobacterium, Lactobacillus, Bacillus, Enterococcus, Escherichia, Streptococcus, Saccharomyces, Streptomyces, and Christensenellaceae. Non-exclusive examples of bacteria that can be used in probiotic compositions described herein include L. acidophilus, Lactobacillus species, such as L. crispatus, L. casei, L. rhamnosus, L. reuteri, L. fermentum, L. plantarum, L. sporogenes, and L. bulgaricus, as well as Bifidobacterum species, such as B. lactis, B. animalis, B. bifidum, B. longum, B. adolescentis, and B. infantis. Yeasts, such as Saccharomyces boulardii, are also suitable as probiotics for administration to the gut, e.g., via oral dosage forms or foods. For example, yogurt is a product which already contains bacteria species, such as Lactobacillus bulgaricus and Streptococcus thermophilus. Yeasts also generate NO from nitrites.

In some embodiments, the composition can include at least about 1% (w/w) of a probiotic bacteria or a combination of probiotic bacteria (e.g., at least about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or more).

The compositions provided herein may be used in methods to modulate the NO, H₂S, and/or CO production of bacterial taxa (e.g., 1, 2, 3, 4, 5 or more taxa) present in the microbiota of a subject. In some embodiments, modulation comprises a change in the structure of the microbiota, such as a change in the relative composition of a taxa or a change in the relative abundance of a taxa, e.g., relative to another taxa or relative to what would be observed in the absence of the modulation. In other embodiments, modulation comprises a change in a function of the microbiota, such as a change in gene expression, level of a gene product (e.g., RNA or protein), or metabolic output of the microbiota, or a change in a functional pathway of the host (e.g., a change in gene expression, level of a gene product, or metabolic output of a host cell or host process).

The methods described herein include administering to a subject a composition in an amount effective to modulate microbiota of the subject. In some embodiments, the abundance of a bacterial taxa may increase relative to other taxa (or relative from one point in time to another) when a composition described herein is administered and the increase can be at least a 5%, 10%, 25% 50%, 75%, 100%, 250%, 500%, 750% increase or at least a 1000% increase. The abundance of a bacterial taxa may also decrease relative to other taxa (or relative from one point in time to another) when a composition described herein is administered and the decrease can be at least a 5%, 10%, 25% 50%, 75%, 85%, 90%, 95%, 96%, 97%, 98%, 99% decrease, or at least a 99.9% decrease.

In some embodiments, a dysbiosis has shifted the microbiota and has increased one or more non-desired taxa and/or increased one or more desired taxa. Administration of the composition can modulate the abundance of the desired and/or non-desired bacterial taxa in the subject's gastrointestinal microbiota, thereby treating the dysbiosis.

The compositions described herein can drive selective changes in both the composition and activity (or function) of the gastrointestinal microbiota by enhancing NO, H₂S, and/or CO production in the microbiota of the subject. For example, the composition can include a selective substrate for one or a limited number of bacteria residing in the GI tract that can produce NO, H₂S, and/or CO. The selective substrate can include, for example, at least one of arginine, nitrate, nitrite, or a salt thereof for stimulating NO production in the microbiota. In other embodiments, the composition administered to the microbiota is capable of altering the gastrointestinal microbiota such that it is higher or lower in specific bacteria.

In some embodiments, the composition administered to the subject can modulate (e.g., increase or decrease) the NO, H₂S, and/or CO production of one or more bacterial taxa residing in the GI tract, such as, e.g., those that belong to genera Bacteroides, Odoribacter, Parabacteroides, Alistipes, Blautia, Clostridium, Coprococcus, Dorea, Eubacterium, Lachnospira, Roseburia, Ruminococcus, Faecalibacterium, Oscillospira, and Subdoligranulum which can be found in the GI tract.

In other embodiments, the composition can modulate (e.g., increase or decrease) the NO, H₂S, and/or CO production of one or more bacterial taxa, such as those that are thought to be associated with a healthy gastrointestinal state, e.g., one or more of the genus Akkermansia, Anaerofilum, Bacteroides, Blautia, Bifidobacterium, Butyrivibrio, Clostridium, Coprococcus, Dialister, Dorea, Fusobacterium, Eubacterium, Faecalibacterium, Lachnospira, Lactobacillus, Phascolarctobacterium, Peptococcus, Peptostreptococcus, Prevotella, Roseburia, Ruminococcus, and Streptococcus, and/or one or more of the species Akkermansia municiphilia, Christensenella minuta, Clostridium coccoides, Clostridium leptum, Clostridium scindens, Dialister invisus, Eubacterium rectal, Eubacterium eligens, Faecalibacterium prausnitzii, Streptococcus salivarius, and Streptococcus thermophilus.

In other embodiments, the composition can modulate (e.g., increase or decrease the NO, H₂S, and/or CO production of one or more bacterial taxa predominantly residing in the small intestine. For example, the composition can modulate the NO, H₂S, and/or CO production one or more (2, 3, 4, 5, 6, 7, 8, 9, 10 or more) bacterial taxa that reside predominantly in the small intestine, such as Actinobacteria, Firmicutes (Bacilli, Clostridia), and Proteobacteria (Alphaproteobacteria, Betaproteobacteria).

In some embodiments, the composition can modulate (e.g., increase or decrease) the NO, H₂S, and/or CO production of one or more bacterial taxa predominantly residing in the large intestine. For example, the composition can modulate the NO, H₂S, and/or CO production of one or more (2, 3, 4, 5, 6, 7, 8, 9, 10 or more) bacterial taxa that reside predominantly in the large intestine, such as Bacteroidetes, Firmicutes (Clostridia), Verrucomicrobia, and Proteobacteria (Deltaproteobacteria).

In other embodiments, the composition can modulate (e.g., increase or decrease) the NO, H₂S, and/or CO production of one or more bacterial taxa predominantly residing in the cecum, such as Actinobacteria, Bacteroides, Bacilli, Clostridia, Mollicutes, Alpha Proteobacteria, and Verrucomicrobia.

In other embodiments, the composition can modulate (e.g., increase or decrease) the NO, H₂S, and/or CO production of one or more bacterial taxa predominantly residing in the ascending colon, such as Actinobacteria, Bacteroides, Bacilli, Clostridia, Fusobacteria, Beta Proteobacteria, Delta/Epsilon Proteobacteria, Gamma Proteobacteria, and Verrucomicrobia.

In some embodiments, the composition can modulate (e.g., increase or decrease) the NO, H₂S, and/or CO production of one or more bacterial taxa predominantly residing in the traverse colon, such as Actinobacteria, Bacteroides, Clostridia, Mollicutes, Fusobacteria, and Gamma Proteobacteria.

In some embodiments, the composition can modulate (e.g., increase or decrease) the NO, H₂S, and/or CO production of one or more bacterial taxa predominantly residing in the descending colon, such as Bacteroides, Clostridia, Mollicutes, Fusobacteria, Delta/Epsilon Proteobacteria and Verrucomicrobia.

In other embodiments, the composition can modulate (e.g., increase or decrease) the NO, H₂S, and/or CO production of one or more bacterial taxa predominantly residing in the sigmoid colon, such as Actinobacteria, Bacteroides, Bacilli, Clostridia, Mollicutes, Alpha Proteobacteria, Beta Proteobacteria, and Verrucomicrobia.

In still other embodiments, the composition can modulate (e.g., increase or decrease) the NO, H₂S, and/or CO production of one or more bacterial taxa predominantly residing in the rectum, such as Bacteroides, Clostridia, Mollicutes, Alpha Proteobacteria, Gamma Proteobacteria, and Verrucomicrobia.

In other embodiments, the composition may modulate the taxa of other microbiota (skin/vagina/mouth).

In some embodiments, the microbes (e.g., bacteria, yeast, etc.) provided in the composition can be genetically modified. For example, genetically modified bacterial strains can include bacteria that are modified to express or over express proteins or enzymes that produce or enhance production of NO, H₂S, and/or CO in the bacteria and/or microbiota of the subject. Proteins or enzymes that can be expressed or overexpressed in the bacteria and/or microbiota of the subject can include nitric oxide synthase or nitrate reductase for NO production, cystathionine γ-lyase, cystathionine β-synthase, and 3-mercaptopyruvate transferase for H₂S production or heme oxygenases (HO-1 and HO-2) for CO production. It will be appreciated that the term “genetically modified”, as used herein indicates any modification of DNA sequences coding for genes involved in the expression of proteins and/or enzymes for regulating NO, H₂S, and/or CO activities including modifications of sequences that regulate the expression of genes coding for such enzymatic activities. Accordingly, genetic modification can be based on construction or selection of mutants of one or more selected bacteria, or it can be based on recombinant DNA-technology.

As used herein, the term “mutant” is used in the conventional meaning of that term; e.g., it refers to strains obtained by subjecting a bacterial strain to any conventionally-used mutagenization treatment including treatment with a chemical mutagen, such as ethanemethane sulphonate (EMS) or N-methyl-N′-nitro-N-nitroguanidine (NTG), UV light or to spontaneously occurring mutants which are selected on the basis of a modified NO, H₂S, and/or CO activity or production. Although it is presently preferred to provide the genetically modified bacteria by random mutagenesis or by selection of spontaneously occurring mutants, i.e., without the use of recombinant DNA technology, mutants of a selected bacteria can be provided by such technology including site-directed mutagenesis and PCR techniques and other in vitro or in vivo modifications of DNA sequences coding for proteins and/or enzymes that regulate NO, H₂S, and/or CO activities or sequences regulating the expression of genes coding for the NO, H₂S, and/or CO activities.

Genetically modified bacteria can also be formed by conventional recombinant DNA-technology including insertion of sequences coding for proteins and/or enzymes regulating NO, H₂S, and/or CO activities, e.g., by replacing a native promoter for such coding sequences by a foreign promoter, which either enhances or reduces the expression of the coding sequences. Moreover, selected bacterial strains can be derived from species that do not have an inherent capability to otherwise produce NO, H₂S, and/or CO or precursors by inserting genes coding for such activities isolated from a different organism comprising such genes. The source of such genes may be bacterial species, yeast species or mammal species. Additionally, genetically modified bacteria can be constructed by modifying metabolic pathways in a bacterium that are not directly involved in NO, H₂S, and/or CO production pathways.

For example, genetically-modified or recombinant bacteria can be modified to suppress or eliminate one or more of the following nitrite reductase genes: nirK, nirB, nirD, nrfF, nrfA, and nrfH.

In some embodiments, genetically-modified or recombinant bacteria can be modified to express one or more of the following nitrate reductase genes: narG, narL, narJ, narQ, narI, napC, napB, napH, napD, napA, napG, and napF.

In some embodiments, genetically-modified or recombinant yeast can be modified to express one or more of the NO synthase genes or cytochrome oxidase genes.

In some embodiments, a method of screening or selectively enhancing a bacteria isolate or mixture for NO, H₂S, and/or CO producing activity can comprise culturing a bacteria sample in a medium comprising a predetermined concentration of a substrate for NO, H₂S, and/or CO producing enzymes; detecting the levels of NO, H₂S, and/or CO in the medium after at least 10 hour of culturing; and selecting the bacteria sample from a medium where the NO, H₂S, and/or CO levels are increased to make a probiotic composition. Screening bacteria can comprise selecting the bacteria sample from a medium where the NO, H₂S, and/or CO levels are increased two-fold after 72 hours, 48 hours, 24 hours, 12 hours, or 6 hours of culturing, or any value there between.

In addition to the above-mentioned bacteria, the compositions or probiotic compositions described herein can include supplemental ingredients. Supplemental ingredients can be metabolic precursors for the bacteria community. In some embodiments, the supplemental ingredient can be an energy substrate, vitamin, or mineral utilized by the bacteria for producing at least one of NO, H₂S, and/or CO.

In some embodiments, such ingredients can include a substrate of nitric oxide synthase or nitrate reductase, arginine, nitrate or a nitrate source, such as an inorganic nitrate source or salt (e.g., calcium nitrate, sodium nitrate, potassium nitrate, and/or magnesium nitrate) or a botanical nitrate source (e.g., beet root, kale, artichoke, holy basil, gymnema sylvestre, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof.)

By way of example, the addition of nitrate permits the bacteria in the composition to efficiently generate NO. In some embodiments, the composition can comprise an amount at or between 5 mg to 20 g of a botanical nitrate source, such as a dehydrated botanical source of the plants listed above. In some embodiments, the composition can comprise 5 mg to 1000 mg or 10 mg to 500 mg of a nitrate salt, or any amount or range there between.

Other supplemental ingredients can include nitrite salts (sodium nitrite, potassium nitrite, calcium nitrite and/or magnesium nitrite. Some embodiments can comprise 1 mg to 100 mg of nitrite salts.

The compositions described herein can be administered to a subject to promote a healthy oral or gut microflora. The diversity of oral and gut microflora can become diminished or altered through the use of antibiotics, mouthwash, and other bactericides. Some diversity, particularly of the NO, H₂S, and/or CO producing bacteria, can be selectively reestablished for preventing or treating (e.g., alleviating one or more symptoms of) medical conditions or disorders ameliorated by NO, H₂S, and/or CO therapy. Such a method comprises administering to a subject a therapeutically effective amount of the composition to modulate the microbiota in the subject.

Disorders treated by the probiotic compositions described herein can include pulmonary disorders associated with hypoxemia and/or smooth muscle constriction in the lungs and airways and/or lung infection and/or lung inflammation and/or lung injury (e.g., pulmonary hypertension, ARDS, asthma, pneumonia, pulmonary fibrosis/interstitial lung diseases, cystic fibrosis, COPD); cardiovascular disease and heart disease (e.g., hypertension, ischemic coronary syndromes, atherosclerosis, heart failure, glaucoma); diseases characterized by angiogenesis (e.g., coronary artery disease); disorders where there is risk of thrombosis occurring; disorders where there is risk of restenosis occurring; inflammatory diseases (e.g., AIDS related dementia, inflammatory bowel disease (IBD), Crohn's disease, colitis, and psoriasis); functional bowel disorders (e.g., irritable bowel syndrome (IBS)); diseases where there is risk of apoptosis occurring (e.g., heart failure, atherosclerosis, degenerative neurologic disorders (Alzheimers, Parkinsons, Huntingtons etc), arthritis, and liver injury (ischemic or alcoholic)); impotence; sleep apnea; diabetic wound healing; cutaneous infections; treatment of psoriasis; obesity caused by eating in response to craving for food; stroke; reperfusion injury (e.g., traumatic muscle injury in heart or lung or crush injury); and disorders where preconditioning of heart or brain for NO protection against subsequent ischemic events is beneficial, central nervous system (CNS) disorders (e.g., anxiety, depression, psychosis, and schizophrenia); and infections caused by bacteria (e.g., tuberculosis, C. difficile infections, among others).

In other embodiments, compositions (with or without supplemental ingredients) can be used to change gene expression in the host to thereby treat diseases and disorders associated with altered gene expression, including but not limited to cancer, heart disease, neurodegenerative conditions, and infections.

In other embodiments, compositions (with or without supplemental ingredients) can be used to change microRNA expression/activity in the host to thereby treat diseases and disorders associated with altered gene expression or protein activity, including but not limited to cancer, heart disease, neurodegenerative conditions, and infections.

In other embodiments, compositions can be used to treat a subject that exhibits at least one symptom or risk of an ischemic tissue or tissue damaged by ischemia. In particular embodiments, the subject is a human who has or who is at risk of having an ischemic tissue or tissue damaged by ischemia, e.g., a subject that has diabetes, peripheral vascular disease, thromboangiitis obliterans, vasculitis, atherosclerosis, arteriosclerosis, cardiovascular disease, coronary artery disease, heart failure, or cerebrovascular disease.

Illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia suitable for treatment or amelioration using the methods described herein, include, ischemia resulting from surgery, chemotherapy, radiation therapy, or cell, tissue, or organ transplant or graft.

In various embodiments, the methods can be used for treating cerebrovascular ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In various embodiments, compositions described herein can be used to treat an ischemic tissue in which it is desirable to increase the blood flow, oxygen supply, glucose supply, or supply of nutrients to the tissue.

By way of example, continuous generation of NO is essential for the integrity of the cardiovascular system and a decreased production and/or bioavailability of NO is central to the development of cardiovascular disorder. In some embodiments, the composition described herein can be administered to a subject to enhance nitric oxide levels in the GI tract or the blood stream. Through the enhancement of nitric oxide levels, cardiovascular disease can be attenuated.

In one embodiment, the compositions described herein can provide a novel therapy for patients experiencing myocardial infarction, stroke, or injury from ischemia-reperfusion insult. Several embodiments provide patients with an extended-release formulation via the administration of compositions containing probiotics and may further contain a substrate of nitric oxide synthase and/or nitrate reductase, among additional components. Such compositions can be administered upon onset of symptoms to provide at least some protection from injury until the patient can be provided with reperfusion therapy, such as in a hospital setting.

Administration of the composition described herein can also lessen the impact of aging on cytoprotective mechanisms. In some embodiments, use of an enteral formulation of the composition in a subject can oppose, attenuate, or reverse NO, H₂S, and/or CO-deficiency-related effects on these mechanisms. In a particular example, attenuation of NO deficiency can be achieved by increasing NO production processes and pathways in a subject, as well as by up-regulating NO processes and pathways.

The probiotic compositions described herein can be formulated for enteral or oral administration to a subject. Forms of the compositions that can be used for oral or enteral administration include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gut (e.g., colon, lower intestine) other than the stomach. All formulations for oral or enteral administration can be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds and/or other agents can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses.

Generally, administered dosages will be effective to deliver femtomolar to micromolar concentrations of the isolated bacteria to the appropriate site, such as the oral cavity, stomach, intestines, or another section of the gastrointestinal tract whether upper or lower. The amount of the described bacteria isolate or mixture that is administered or prescribed to the subject can be about, at least about, or at most about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 g, or any range derivable therein. In some embodiments, the probiotic composition comprises 0.1 g to 10 g or 1 g to 5 g of bacteria isolate or mixture. In some embodiments, the composition can comprise a bacteria isolate or mixture as described with an activity of 0.5 to 100 billion colony forming units or 5 billion to 20 billion colony forming units or 8 billion to 12 billion colony forming units. When provided in a discrete amount, each intake of described bacteria or composition comprising the bacteria can be considered a “dose” and present in a dosage form. A medical practitioner may prescribe or administer multiple doses over a particular time course (treatment regimen) or indefinitely.

Dosage forms can be formulated to affect the viability of the probiotics upon administration, as well as the rate, extent, and duration of probiotic activity. In some embodiments, the oral formulations described herein are formulated to be “controlled release” formulations. “Controlled release”, as used herein, signifies a release of an active agent or ingredient from a formulation in the gastrointestinal tract of the subject.

In some embodiments, the composition further comprises a carrier. As used herein, the term “carrier” refers to a typically inert substance used as a diluent or vehicle for a drug such as a therapeutic agent. The term also encompasses a typically inert substance that imparts cohesive qualities to the composition and can also enhance the viability or activity of the probiotics, particularly if freeze dried. Suitable carriers can be lyoprotectants and matrix forming additives that protect the bacteria during a freeze-drying process.

Examples of lyoprotectants and matrix forming additives comprise albumin, mannitol, sucrose, betaine, gum acacia, and trehalose. Other carriers for use in the compositions described herein include, without limitation, a solid, semi-solid, or liquid such as a binder or a gum base. Non-limiting examples of binders include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, VEEGUM, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof.

These binders can be pre-processed to improve their flowability and taste by methods known in the art such as freeze drying [see, e.g., “Fundamentals of Freeze-Drying,” Pharm. Biotechnol., Vol. 14, pp. 281-360 (2002); “Lyophililization of Unit Dose Pharmaceutical Dosage Forms,” Drug. Dev. Ind. Pharm., Vol. 29, pp. 595-602 (2003)]; solid-solution preparation; and lubricant dusting and wet-granulation preparation with a suitable lubricating agent (see, e.g., Remington: The Science and Practice of Pharmacy, supra). For example, MANNOGEM and SORBOGEM, sold by SPI Pharma Group (New Castle, Del.), are freeze-dried, processed forms of mannitol and sorbitol, respectively. Typically, when a binder is included in the formulation, the compositions described herein comprise from about 15% to about 90% by weight of the binder, and preferably from about 35% to about 80%. However, one skilled in the art will appreciate that the compositions described herein can be made without any binders, e.g., to produce a highly friable dosage form.

Bacteria can be formulated for combination with a liquid carrier moments prior to administration, such as a reconstitutable powder. The liquid carrier can be water, and can be provided at room temperature or slightly warmer. The reconstitutable powder composition can further comprise an energy substrate to facilitate activation of the bacteria.

In other embodiments, the carrier can comprise a gum base. Non-limiting examples of gum bases include materials selected from among the many water-insoluble and saliva-insoluble gum base materials known in the art. For example, in some instances, the gum base comprises at least one hydrophobic polymer and at least one hydrophilic polymer. Non-limiting examples of hydrophobic and hydrophilic polymers for gum bases include both natural and synthetic polymers such as elastomers, rubbers, and combinations thereof. Examples of suitable natural polymers include, without limitation, substances of plant origin such as chicle, jelutong, gutta percha, crown gum, and combinations thereof. Examples of suitable synthetic polymers include elastomers such as butadiene-styrene copolymers, isobutylene and isoprene copolymers (e.g., “butyl rubber”), polyethylene, polyisobutylene, polyvinylester (e.g., polyvinyl acetate and polyvinyl acetate phthalate), and combinations thereof. In other instances, the gum base comprises a mixture of butyl rubber (i.e., isobutylene and isoprene copolymer), polyisobutylene, and optionally, polyvinylacetate (e.g., having a molecular weight of approximately 12,000). Typically, the gum base comprises from about 25% to about 75% by weight of these polymers, and preferably from about 30% to about 60%.

Other carriers can include lubricating agents; wetting agents; emulsifying agents; solubilizing agents; suspending agents; preserving agents, such as methyl-, ethyl-, and propyl-hydroxy-benzoates, butylated hydroxytoluene, and butylated hydroxyanisole; sweetening agents; flavoring agents; coloring agents; and disintegrating agents (i.e., dissolving agents) such as crospovidone as well as croscarmellose sodium and other cross-linked cellulose polymers. Lubricating agents can be used to prevent adhesion of the dosage form to the surface of the dies and punches, and to reduce inter-particle friction. Lubricating agents may also facilitate ejection of the dosage form from the die cavity and improve the rate of granulation flow during processing. Examples of suitable lubricating agents include, without limitation, magnesium stearate, calcium stearate, zinc stearate, stearic acid, simethicone, silicon dioxide, talc, hydrogenated vegetable oil, polyethylene glycol, mineral oil, and combinations thereof. The compositions of the present invention can comprise from about 0% to about 10% by weight of the lubricating agent, and preferably from about 1% to about 5%.

In other embodiments, the carrier can comprise one or more sweetening agents. Sweetening agents can be used to improve the palatability of the composition by masking any unpleasant tastes it may have. Examples of suitable sweetening agents include, without limitation, compounds selected from the saccharide family such as the mono-, di-, tri-, poly-, and oligosaccharides; sugars such as sucrose, glucose (corn syrup), dextrose, invert sugar, fructose, maltodextrin, and polydextrose; saccharin and salts thereof such as sodium and calcium salts; cyclamic acid and salts thereof; dipeptide sweeteners; chlorinated sugar derivatives such as sucralose and dihydrochalcone; sugar alcohols such as sorbitol, sorbitol syrup, mannitol, xylitol, hexa-resorcinol, and the like, and combinations thereof. Other suitable sweeting agents may include natural plant-based sweeteners such as stevia. Hydrogenated starch hydrolysate, and the potassium, calcium, and sodium salts of 3,6-dihydro-6-methyl-1 1,2,3-oxathiazin-4-one-2,2-dioxide may also be used. Of the foregoing, sorbitol, mannitol, and xylitol, either alone or in combination, are preferred sweetening agents. The compositions described herein can include from about 0% to about 80% by weight of the sweetening agent, from about 5% to about 75%, or from about 25% to about 50%.

In still other embodiments, the carrier can comprise one or more flavoring agents. Flavoring agents can also be used to improve the palatability of the composition. Examples of suitable flavoring agents include, without limitation, natural and/or synthetic (i.e., artificial) compounds such as peppermint, spearmint, wintergreen, cinnamon, menthol, cherry, strawberry, watermelon, grape, banana, peach, pineapple, apricot, pear, raspberry, lemon, grapefruit, orange, plum, apple, fruit punch, passion fruit, chocolate (e.g., white, milk, dark), vanilla, caramel, coffee, hazelnut, combinations thereof, and the like.

In some embodiments, the carrier can comprise one or more coloring agents. Coloring agents can be used to color code the composition, for example, to indicate the type and dosage of the bacteria therein. Coloring agents include, without limitation, natural and/or artificial compounds such as FD & C coloring agents, natural juice concentrates, pigments such as titanium oxide, silicon dioxide, and zinc oxide, combinations thereof, and the like. The compositions of the present disclosure can comprise from about 0% to about 10% by weight of the flavoring and/or coloring agent, preferably from about 0.1% to about 5%, and more preferably from about 2% to about 3%.

The dosage form to be administered will, in any event, contain a quantity of the bacteria in a therapeutically effective amount for developing a particular microflora (at least temporarily) within a region of the gastrointestinal tract. In addition, the dosage form can contain a therapeutically effective amount of supplemental ingredients to promote the therapeutic function of the bacteria, e.g., to produce NO, H₂S, and/or CO.

In some embodiments, the composition can include freeze-dried bacteria. The described bacteria can be cultured in liquid medium, and then collected by centrifugation to remove the liquid medium. Alternatively, cells can be harvested from agar plates.

The collected bacteria can then be suspended in volume of lyophilization medium (e.g., in equal volumes) that comprises a lyoprotectant(s) and a matrix agent(s) that allow the sample to retain its shape during and after processing. Disaccharides such as sucrose and trehalose can be used as lyoprotectants. Matrix forming additives, often referred to as excipients, include mannitol, BSA, serum, and skim milk. This bacteria/lyophilization medium mixture can be partitioned into aliquots and transferred into a sterile vessel to undergo the lyophilization process.

Techniques of lyophilization are known or will be apparent to those skilled in the art. Examples are described in Guergoletto et al. (2012). “Dried Probiotics for Use in Functional Food Applications—Methods and Equipment,” ISBN: 978-953-307-905-9, InTech, Available from: http://www.intechopen.com/books/food-industrialprocesses-methods-and-equi-pment/dried-probiotics-for-use-in-functional-food-applications and Gitaitis, “Refinement of Lyophilization Methodology for Storage of Large Numbers of Bacterial Strains” Plant Disease, 71: 615-616 (1987), which are hereby incorporated by reference.

The compositions described herein can be administered to a subject via any of the accepted modes of administration to the gastrointestinal tract of a subject. It should be noted that any method of delivery that delivers the bacteria and any supplemental ingredients to a suitable section of the gastrointestinal tract can be utilized. In particular, any method that would deliver the bacteria as well as any supplemental ingredients to the microbiota of the subject where they can begin to act therapeutically can be utilized. Such delivery formulations including but not limited to suppositories (both rectal and vaginal), sprays (both oral and nasal), subdermal implants, and controlled release capsules that allow the formulation to move past the stomach region of the patient, e.g., pH controlled release capsules.

The probiotic composition may be administered 1, 2, 3, 4, 5, 6, 7, 8, 9, 10, 11, 12, 13, 14, 15, 16, 17, 18, 19, 20, 30, 40, 50, 60, 70, 80, or more times or any range derivable therein. It is further contemplated that the dose may be taken for an indefinite period of time or for as long as the subject exhibits symptoms of the medical condition for which the described bacteria was prescribed or to prevent or inhibit such conditions. Also, the dose may be administered every 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23.24 hours, or 1, 2, 3, 4, 5, 6, 7 days, or 1, 2, 3, 4, 5 weeks, or 1, 2, 3, 4, 5, 6, 7, 8, 9, 10, 11, 12 months or more, or any range derivable therein. Alternatively, it may be administered systemically over any such period of time and be extended beyond more than a year

Other embodiments described herein relate to measuring the NO, H₂S, and/or CO levels in the subject being treated before and/or after probiotic compositions described have been administered. For example, a method of measuring levels of NO can comprise administering a probiotic composition to a subject as described herein and measuring the levels of NO in the saliva or breath of the subject after 10 minutes to 4 hours or 1 hour to 3 hours or 30 minutes to 2 hours of the administration and/or prior to administration.

In some embodiments, the compositions described herein can be administered in a combinatorial therapy or combination therapy that includes administration of the composition with one or more additional active agents. The phrase “combinatorial therapy” or “combination therapy” embraces the administration of the compositions, and one or more therapeutic agents as part of a specific treatment regimen intended to provide beneficial effect from the co-action of these therapeutic agents. Administration of these therapeutic agents in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

In some embodiments, the compositions described herein can be administered in combination with active agents, such as vasodilators, prostanoid agonists, antiandrogens, cyclosporins and their analogues, triterpenes, alone or as a mixture. The vasodilators can include potassium channel agonists including minoxidil and its derivatives, aminexil and the compounds described in U.S. Pat. Nos. 3,382,247, 5,756,092, 5,772,990, 5,760,043, 5,466,694, 5,438,058, 4,973,474, chromakalin and diazoxide. The antiandrogens can include 5α-reductase inhibitors such as finasteride and the compounds described in U.S. Pat. No. 5,516,779, cyprosterone acetate, azelaic acid, its salts and its derivatives, and the compounds described in U.S. Pat. No. 5,480,913, flutamide and the compounds described in U.S. Pat. Nos. 5,411,981, 5,565,467 and 4,910,226. The anti-inflammatory agents can include inhibitors specific for Cox-2 such as for example NS-398 and DuP-697 (B. Batistini et al., DN&P 1994; 7(8):501-511) and/or inhibitors of lipoxygenases, in particular 5-lipoxygenase, such as for example zileuton (F. J. Alvarez & R. T. Slade, Pharmaceutical Res. 1992; 9(11):1465-1473).

Other active compounds, which can be present in pharmaceutical compositions can include aminexil and its derivatives, 60-[(9Z,12Z)octadec-9,12-dienoyl]hexapyranose, benzalkonium chloride, benzethonium chloride, phenol, oestradiol, chlorpheniramine maleate, chlorophyllin derivatives, cholesterol, cysteine, methionine, benzyl nicotinate, menthol, peppermint oil, calcium panthotenate, panthenol, resorcinol, protein kinase C inhibitors, prostaglandin H synthase 1 or COX-1 activators, or COX-2 activators, glycosidase inhibitors, glycosaminoglycanase inhibitors, pyroglutamic acid esters, hexosaccharidic or acylhexosaccharidic acids, substituted ethylenearyls, N-acylated amino acids, flavonoids, derivatives and analogues of ascomycin, histamine antagonists, triterpenes, such as ursolic acid and the compounds described in U.S. Pat. Nos. 5,529,769, 5,468,888, 5,631,282, saponins, proteoglycanase inhibitors, agonists and antagonists of oestrogens, pseudopterins, cytokines and growth factor promoters, IL-1 or IL-6 inhibitors, IL-10 promoters, TNF inhibitors, vitamins, such as vitamin D, analogues of vitamin B12 and panthotenol, hydroxy acids, benzophenones, esterified fatty acids, and hydantoin.

In one embodiment, the compositions described herein can be administered in combination with an agent that imposes nitrosative or oxidative stress. Agents for selectively imposing nitrosative stress to inhibit proliferation of pathologically proliferating cells in combination therapy with the probiotic composition and dosages and routes of administration therefore include those disclosed in U.S. Pat. No. 6,057,367, which is incorporated herein by reference in its entirety. Supplemental agents for imposing oxidative stress (i.e., agents that increase GSSG (oxidized glutathione) over GSH (glutathione) ratio or NAD(P) over NAD(P)H ratio or increase thiobarbituric acid derivatives) in combination therapy with the AKR inhibitors (e.g., imiristat) or GSNOR/ADH inhibitors (e.g., N6022) or GSH inhibitors including, for example, L-buthionine-S-sulfoximine (BSO), thioredoxin reductase inhibitors (e.g. auranofin or arsenicals) glutathione reductase inhibitors (e.g., BCNU), inhibitors or uncouplers of mitochondrial respiration, and drugs that increase reactive oxygen species (ROS), e.g., adriamycin, in standard dosages with standard routes of administration.

The composition described herein may also be co-administered with a phosphodiesterase inhibitor (e.g., rolipram, cilomilast, roflumilast, VIAGRA (sildenifil citrate), CLALIS (tadalafil), LEVITRA (vardenifil), etc.), a β-agonist, a steroid, or a leukotriene antagonist (LTD-4). Those skilled in the art can readily determine the appropriate therapeutically effective amount depending on the disorder to be ameliorated.

The therapeutically effective amount for the treatment of a subject afflicted with a disorder ameliorated by microbiota NO, H₂S, and/or CO donor therapy is an amount in vivo that causes amelioration of the disorder being treated or protects against a risk associated with the disorder. For example, for asthma, a therapeutically effective amount is a bronchodilating effective amount; for cystic fibrosis, a therapeutically effective amount is an airway obstruction ameliorating effective amount; for ARDS, a therapeutically effective amount is a hypoxemia ameliorating effective amount; for heart disease, a therapeutically effective amount is an angina relieving or angiogenesis inducing effective amount; for hypertension, a therapeutically effective amount is a blood pressure reducing effective amount; for ischemic coronary disorders, a therapeutic amount is a blood flow increasing effective amount; for atherosclerosis, a therapeutically effective amount is an endothelial dysfunction reversing effective amount; for glaucoma, a therapeutic amount is an intraocular pressure reducing effective amount; for diseases characterized by angiogenesis, a therapeutically effective amount is an angiogenesis inhibiting effective amount; for disorders where there is risk of thrombosis occurring, a therapeutically effective amount is a thrombosis preventing effective amount; for disorders where there is risk of restenosis occurring, a therapeutically effective amount is a restenosis inhibiting effective amount; for chronic inflammatory diseases, a therapeutically effective amount is an inflammation reducing effective amount; for disorders where there is risk of apoptosis occurring, a therapeutically effective amount is an apoptosis preventing effective amount; for impotence, a therapeutically effective amount is an erection attaining or sustaining effective amount; for obesity, a therapeutically effective amount is a satiety causing effective amount; for stroke, a therapeutically effective amount is a blood flow increasing or a TIA protecting effective amount; for reperfusion injury, a therapeutically effective amount is a function increasing effective amount; and for preconditioning of heart and brain, a therapeutically effective amount is a cell protective effective amount, e.g., as measured by troponin or CP.

### Example

In this Example, we examined the interplay of microbiota on the regulation of host cellular function. The nematode C. elegans was selected as the host it provides a tractable and elegant system for investigating the interplay between commensal bacteria and a host animal.

We theorized we could test the idea of a general mechanism of interspecies signaling by identifying proteome-wide changes in the host organism that are mediated by commensal bacteria. Nitric oxide (NO) mediated S-nitrosylation of cysteine residues provides a unique opportunity to test these ideas. It is estimated that ˜70% of the universal proteome may be subject to post-translational regulation by S-nitrosylation (˜7000 proteins reported to date), primarily at conserved sites, including effects on protein activity, stability, localization and interactions. S-nitrosylation thus operates across phylogeny as a fundamental mechanism for regulating protein function, thereby controlling diverse physiology including motility, metabolism, energy utilization and lifespan. Notably, many members of the native nematode microbiota (e.g., B. subtilis) are capable of producing NO, which has also been linked to C. elegans lifespan.

Bacterial NO production is primarily dependent on the activity of two enzymes: NO synthase (NOS) and/or nitrate reductase (NarG). These enzymes are therefore prime candidates for mediating protein S-nitrosylation in C. elegans. Importantly, C. elegans are known to be reliant on commensal bacteria as a source for NO. We reasoned, therefore, that microbe-generated NO might potentially influence nematode physiology broadly via modification of C. elegans proteins. Here, by selectively eliminating NO generation in the microbiota and its S-nitrosylation of nematode proteins, we reveal a general mechanism by which the microbiota post-translationally shapes the proteome of its host to regulate cellular function and physiology. More specifically, our studies reveal thousands of proteins targeted by interspecies S-nitrosylation, exemplified by bacterial S-nitrosylation of C. elegans Argonaute proteins to regulate RISC assembly, miRNA activity and developmental timing.

### Experimental Model and Subject Details

C. elegans Strains, Maintenance and Preparation

Wild-type N2 Bristol and mutant let-7(n2853) strains were obtained from the Caenorhabditis Genetics Center (CGC, University of Minnesota, Minneapolis, Minn., USA). N2 nematodes were maintained and prepared using standardized methods including nematode growth medium supplemented with 1 mM arginine, and age synchronization by hypochlorite.

All strains were out-crossed at least 3 times with wild-type nematodes. The let-7(n2853) strains were maintained at 15° C., and were incubated at 15° C., 21° C. or 25° C. in specific experiments as indicated.

### Bacterial Strains

subtilis strain 1A1 (strain 168) and the isogenic Δnos, were obtained from the Bacillus Genetic Stock Center (BGSC) at The Ohio State University. The Δnos strain had been described previously (Koo et al., 2017). These bacteria were grown in LB medium at 37° C. Erythromycin (20 μg/ml) was added to the media when growing the Δnos B. subtilis. E. coli strain BW25113 WT and ΔnarG were procured from the KEIO collection from the Coli Genetic Stock Center (CGSC) at Yale University. E. coli were grown in LB medium at 37° C.

### Cell Lines

HEK293 cells (female) and HeLa cells (female) procured from ATCC (Manassas, Va.) were already authenticated using STR profiling. Cultured cells were grown in 1×DMEM (Life Technologies, Carlsbad, Calif.) supplemented with Fetal Bovine Serum (Sigma-Aldrich, St. Louis, Mo.) to a final concentration of 10% plus 1% Antibiotic-Antimycotic solution (Life Technologies, Carlsbad, Calif.).

### Method Details

### Reagents

S-nitrosocysteine (CysNO) was prepared as previously described. DETA-NONOate (DETA-NO) was obtained from Cayman Chemicals (Ann Arbor, Mich.) and was prepared and used per manufacturer's instructions. All other chemicals were obtained from Sigma-Aldrich (St. Louis, Mo.). Antibodies for Western blotting included mouse monoclonal anti-FLAG M2 (Sigma-Aldrich, St. Louis, Mo.), goat anti-DAF-16 (Santa Cruz Biotechnology, Dallas, Tex.), polyclonal rabbit anti-His-tag and monoclonal rabbit anti-AGO2 (Cell Signaling Technology, Danvers, Mass.), rabbit polyclonal anti-ALG-1 (ThermoFisher Scientific, Waltham, Mass.), GW182 antibody (Novus Biologicals, Littleton, Colo.), c-myc antibody (R&D systems, Minneapolis, Minn.) and DAF-16 antibody (Santa Cruz Biotechnology, Dallas, Tex.). SilverQuest™ silver staining kit was procured from Invitrogen™ (ThermoFisher Scientific, Waltham, Mass.) and Imperial™ Protein Stain was from ThermoFisher Scientific (Waltham, Mass.). AIN-1 antibody was a gift from John K. Kim (Johns Hopkins University)

### Plasmids

To express 6×His tagged recombinant proteins, the amplified AGO2 cDNA was cloned into pET21b vector (Novagen, Merck Biosciences) and sequenced (pET21b-AGO2-His). The sequences of the primers are given in the Key Resources Table. FLAG-AGO2, Hmga2 3′UTR WT luciferase and Hmga2 3′UTR m7 luciferase were obtained from Addgene.

### Elegans Bursting Assays

Let-7(n2853) worms possessing either WT-ALG-1 or C855S-ALG-1 were synchronized by hypochlorite and grown at either 15° C., 21° C. or 25° C. The number of animals dying through vulval rupture were counted and compared against the number of surviving animals.

C. elegans CRISPR Genome Editing

Genome editing was performed using CRISPR/Cas9 as described (Paix et al., 2015). Briefly, purified Cas9 (NEB), tracrRNA, dpy-10 and alg-1 crRNA, and repair templates were incubated at 37° C. for ten minutes for in vitro assembly, then loaded for injection. Worms were screened for rollers, bred to separate dpy-10 from alg-1 edits, and sequenced. TracrRNA was purchased from Dharmacon (Lafayette, Colo.).

C. elegans Lysis

All steps were performed at 4° C. unless stated otherwise.

### For Protein Extraction

Worms were lysed in 1 ml of HEN buffer (100 mM HEPES, 1 mM EDTA, 0.1 mM Neocuproine) containing protease inhibitors by repeatedly snap freezing in liquid nitrogen/thawing in 37° C. water bath. Worms in HEN buffer were sonicated, employing four 15 second pulses at setting 4 of the VirSonic sonicator (VirTis, SP Industries, Warminster, Pa.). After sonication, the lysate was visualized under the microscope to confirm worm rupture.

### For RNA Extraction

Worms were washed four times in M9 buffer, then resuspended in 1 ml of QIAzol reagent. They were then subjected to at least two repeated cycles of snap freeze/thaw in liquid nitrogen/37° C. water bath. Subsequently, they were disrupted at room temperature in TissueLyser II (Qiagen, Hilden, Germany) using a stainless steel bead in each sample tube for 2 minutes at 30 Hz. Total RNA was extracted in QIAzol per manufacturer's instructions.

### Detection of S-Nitrosylated Proteins by SNO-RAC (SNO Resin-Assisted Capture)

S-nitrosylated proteins were isolated by SNO-RAC as described. In brief, cells were lysed in HEN buffer additionally containing 1% NP-40, 50 mM NaCl and protease inhibitors. Free cysteines were blocked with S-Methyl methanethiosulfonate (MMTS). After acetone precipitation and multiple 70% acetone washes, proteins were re-suspended in HEN buffer containing 1% SDS. 50 μl thiopropyl Sepharose 6B resin (GE, Chicago, Ill.) and 50 mM sodium ascorbate were added, followed by rotation in the dark for 4 hr at room temperature. Following multiple washes, the bound proteins were eluted in 2×SDS-PAGE loading buffer containing 10% β-mercaptoethanol. Following separation on reducing pre-cast 4-20% SDS-PAGE gels (Bio-Rad Laboratories, Hercules, Calif.), individual SNO-proteins were detected by Western blotting using specific antibodies (anti-FLAG, anti-myc, anti-ALG-1, anti-AGO2, anti-GW182) or the gel was stained using the SilverQuest™ silver staining kit (ThermoFisher Scientific, Waltham, Mass.) or Imperial™ Protein Stain (ThermoFisher Scientific, Waltham, Mass.), per manufacturer instructions.

### Colony Forming Unit Assays

Individual C. elegans were lysed in a bead-beater (BioSpec Products Bartlesville, Okla.) at the highest setting, using 1 mm Zirconia beads (BioSpec Products Bartlesville, Okla.) with 5 l-min cycles of beating alternating with 1-min cooling intervals, followed by centrifugation at 12,000 g for 1 minute at 4° C. The supernatant was then dilution plated on LB plates and the resulting colonies were counted after an overnight incubation at 37° C.

### Reverse Transcription and Real-Time PCR

RNA was extracted using the QIAzol lysis reagent (Qiagen, Hilden, Germany) per manufacturer's instructions. 2 μg of RNA was treated with RQ1 RNase-free DNase (Promega, Madison, Wis.), per manufacturer's instructions. Cleanup after the DNAase treatment was performed using phenol-chloroform extraction and ethanol precipitation and the RNA was finally resuspended in nuclease free water. The cDNA was prepared using the 5× iScript RT Supermix (Bio-Rad, Hercules, Calif.) per manufacturer's instructions. Gene specific primers were used for real-time PCR in an Applied Biosystems StepOnePlus instrument using either 2×iQ SYBR green supermix (BioRad, Hercules, Calif.) for detecting mRNAs or specific Applied Biosystems TaqMan Gene Expression Assays (ThermoFisher Scientific, Waltham, Mass.) for detecting microRNAs. For RNA from C. elegans the expression of Y45 mRNA in each sample was used to normalize the expression of lin-41 mRNA, while the levels of U18 RNA was used to normalize the expression of the let-7 micro RNA. For RNA from cultured human cells, U6 RNA and 5S rRNA were used to normalize the expression for microRNAs and mRNAs, respectively. Fold-change in expression was calculated using the ΔΔCT method. Real-time PCR primers for lin-41 and Y45 from C. elegans have been validated previously.

### Purification of 6×His Tagged Recombinant Proteins

The AGO2 cDNA was cloned into the pET21b (Novagen) vector to introduce a 6×His tag at its C-terminus. Transformed overnight bacterial cultures were sub-cultured into 3 L of LB medium at 4% volume. At OD₆₀₀ of 0.4, cultures were induced by addition of 100 μM IPTG and grown further for 4 hr at 25° C. Cells were harvested by centrifugation at 4500×g for 15 min. For every 1 L of culture, bacterial pellets were lysed in 2 mL of 2× Cellytic B cell lysis buffer (Sigma-Aldrich, St. Louis, Mo.) and 2 mL of buffer containing 50 mM NaH₂PO₄, 300 mM NaCl, 0.2 mg/ml lysozyme, 5 μg/ml DNase and 1 mM PMSF. To aid the lysis, rotation at room temperature was performed for 30 min and then the supernatant was collected after centrifugation at 16,000×g for 12 min.

The collected lysate was diluted 4-fold in a buffer containing 50 mM NaH₂PO₄, 300 mM NaCl, 10 mM imidazole and incubated at room temperature for 2 hr with rotation with 1.5 ml of Ni-NTA agarose (pre-equilibrated with the 50 mM NaH₂PO₄, 300 mM NaCl buffer). This slurry was then poured into empty PD-10 columns (GE Healthcare, Chicago, Ill.). Beads were then washed with 100 ml of 50 mM NaH₂PO₄, 300 mM NaCl buffer containing 20 mM imidazole. Elution was done using 20 ml of 50 mM NaH₂PO₄, 300 mM NaCl buffer with 250 mM imidazole, with 1 ml fractions collected. 15 μl from each collected fraction was analyzed by SDS-PAGE. Fractions containing the pure AGO2 protein were pooled and stored at −80° C. in 30% glycerol.

### Mass Spectrometric Identification of AGO2 S-Nitrosylation Site

200 μg of purified 6×His-tagged AGO2 was further purified by immunoprecipitation using 10 μg of AGO2 specific antibody (Cell Signaling Technologies, Danvers, Mass.) at 4° C. overnight with rotation, in a final volume of 600 μl of elution buffer containing 50 mM NaH₂PO₄, 300 mM NaCl buffer with 250 mM imidazole, after which 30 μl of protein A/G Sepharose (Pierce, ThermoScientific, Waltham, Mass.) was added and the samples were incubated for 2 hours at 4° C. The protein A/G Sepharose-antibody complexes were pulled down by centrifugation at 1000×g in a swinging bucket rotor. Following five washes with the IP/lysis buffer (ThermoScientific, Waltham, Mass.) at 1000×g for 1 min each, the bound proteins were eluted using Gentle Elution buffer (ThermoScientific, Waltham, Mass.). The eluate was then treated with 100 m CysNO for 30 minutes, in a final volume of 300 μl of sodium-phosphate buffer (pH 7.4). They were then precipitated using 3× volume of acetone in the presence of 100 μg of BSA carrier protein. After 3 washes with 70% acetone, the pellet was re-suspended in a final volume of 600 μl HEN buffer containing 1% SDS and 100 mM N-ethylmaleimide and was incubated at 20° C. for 25 minutes.

The samples were then re-precipitated with acetone and washed 3 times with 70% acetone to remove excess maleimide. Pellets were then resuspended in 800 l of HEN buffer with 1% SDS and were divided in two equal aliquots based upon volume. One aliquot was reduced in the presence of 500 mM ascorbate and 500 mM IAA, while the other aliquot was treated the same way except without the addition of ascorbate. The samples were then passed through a 50 kDa size cut off filter (Amicon, Millipore-Sigma, Burlington, Mass.) and run on a 4-20% polyacrylamide gel, followed by staining with Imperial reagent, as described earlier.

Gel bands were sliced and washed with 50% acetonitrile/50% ammonium bicarbonate, while vortexing at room temperature for more than 5 hrs. After removal of washing buffer, 200 μl of 100% acetonitrile was added to dehydrate gel bands for 10 min. Gel pieces were completely dried by vacuum spin dryer at room temperature for 10 min. Dry gel pieces were incubated with 200 μl of 10 mM DTT for 45 min at 37° C. After removal of DTT solution, 200 μl of 55 mM N-methylmaleimide was added to gel pieces at 37° C. for 45 min. 200 μl of 100 mM ammonium bicarbonate and 200 μl of 100% acetonitrile were used alternatively to wash the gel bands (vortexing time=10 min for each wash). Gel pieces were then dried by vacuum spin dryer at room temperature for 10 min and incubated with enzyme solution containing 500 ng sequencing grade trypsin in 100 mM ammonium bicarbonate buffer at 37° C. overnight. Supernatant containing protein digests was transferred to a 1.5 ml tube. Peptides were extracted by incubating the gel pieces in 60% acetonitrile containing 5% formic acid for 30 minutes with constant vortexing, followed by sonication for 15 min. This was repeated three times and the extracts were pooled in a 1.5 ml tube and dried to completion in a speed-vac. The dried peptides were then reconstituted by 0.1% formic acid and subjected to LC/MS (liquid chromatography/mass spectrometry) analysis.

A UPLC (Waters, Milford, Mass.) coupled with an Orbitrap Elite hybrid mass spectrometer (ThermoScientific, Waltham, Mass.) was used to separate and identify peptides. Peptides were loaded to a 5-cm×75-μm Pico Frit C18 column (New Objective, Woburn, Mass.) and emitted to the mass spectrometer by a 10 μm nanospray emitter (New Objective, Woburn, Mass.). A linear chromatography gradient from 1% to 40% organic phase, using 100% acetonitrile as organic phase and 0.1% formic acid as inorganic phase, was used to separate peptides for 60 minutes at a flow rate of 0.3 μl/min. All mass spectrometry data were acquired in positive ion mode. A full scan at resolution of 120,000 was conducted followed by 20 tandem MS/MS scans. CID cleavage mode was performed at normalized collision energy of 35%.

MS data were searched against the human AGO2 protein sequence and its reversed sequence as a decoy. Massmatrix database was used for data searching. Modifications including oxidation of methionine and labeling of cysteine (NMM, NEM or IAA) were used as variable modifications for performing the search. Trypsin was selected as an in silico enzyme to cleave proteins after Lys and Arg. Precursor ion searching was within 10 ppm mass accuracy and product ions within 0.8 Da for CID cleavage mode. After identification by software, manual matching of each product ions in mass spectrometry was applied for confirmation.

Mass Spectrometric Identification of C. elegans SNO-Proteome

All liquid reagents used were HPLC quality grade. Protein digestion was performed with trypsin (Promega, Madison, Wis.) in 50 mM ammonium bicarbonate buffer by incubation overnight at 37° C. For MS analysis the data were collected by a high-resolution MS² method using an Orbitrap Fusion mass spectrometer (Thermo Fisher Scientific, Waltham MS) coupled to a ProxeonEASY-nLC 1000 liquid chromatography (LC) system (Thermo Fisher Scientific, Waltham, Mass.). The run sequence used high-resolution measurements for MS¹ and MS² in the Orbitrap. The capillary column was a 100 m inner diameter microcapillary column packed with −35 cm of Accucore C18 resin (2.6 m, 150 Å, Thermo Fisher Scientific). Peptides were separated in 240 minute acidic acetonitrile (AcN) gradients by LC prior to MS injection. The scan sequence began with a MS¹ spectrum (Orbitrap analysis; resolution 120,000; mass range 400-1400 Th). MS² analysis in the Orbitrap (resolution was 15,000 at 200 Th) followed collision-induced dissociation (CID, CE=35) with a maximum ion injection time of 200 ms and an isolation window of 0.7 Da. Peptides were searched using a SEQUEST-based in-house software against the C. elegans proteome database with a target decoy database strategy. Spectra were converted to mzXML using a modified version of ReAdW.exe. Searches were performed using a 50 ppm precursor ion tolerance for total protein level profiling. The product ion tolerance was set to 0.03 Da. Oxidation of methionines and modification of cysteines (+15.9949146221 Da and +45.987721 Da, respectively) were set as variable modifications. Peptide-spectrum matches (PSMs) were identified, quantified and collapsed to a 1% peptide false discovery rate (FDR) and then collapsed further to a final protein-level FDR of 1%.

### NO Donor Treatment and Lysis of Cultured Cells

Transfected HEK293 or HeLa cells were cultured at 37° C. in 15 cm tissue culture-treated dishes. They were then treated with either 500 μM DETA-NOate (t_{1/2}=20 hr) for 16 hours, 100 μM CysNO for 10 min, or 200 μM dipropylenetriamine (DPTA)-NONOate (Cayman Chemicals, Ann Arbor, Mich.) for 6 hours. Cells were washed once with DPBS without calcium chloride and magnesium chloride from Gibco® (Life Technologies, Carlsbad, Calif.) and incubated on ice for 10 minutes in IP lysis/wash buffer (ThermoFisher Scientific, Waltham, Mass.) with added EDTA-free protease inhibitor tablet (Roche, Basel, Switzerland). This was followed by centrifugation at 14,000 g for 10 minutes to collect the supernatant. The lysates were then used for immunoprecipitation experiments.

### Immunoprecipitation Experiments

For HEK293 cells, all steps were performed either on ice or in the cold room at 4° C. Immunoprecipitations (IP) were performed with 3 mg of total lysate that was pre-cleared using a Pierce™ control agarose resin (ThermoFisher Scientific, Waltham, Mass.), after which 10 μg of IP antibody was added, and samples were incubated overnight at 4° C. with rotation. The next day, 30 μl of protein A/G Sepharose (ThermoFisher Scientific, Waltham, Mass.) was added and the samples were incubated for 2 hours at 4° C. The protein A/G Sepharose-antibody complexes were pulled down by centrifugation at 1000 g in a swinging bucket rotor. Following five washes with the IP lysis/wash buffer, the bound proteins were eluted at room temperature in glycine-HCL buffer, pH 3.5. The eluates were neutralized by adding 3 μl of 1M Tris-HCl, pH 8.0 and analyzed by western blotting. FLAG-AGO2 was a gift from Edward Chan (Addgene plasmid #21538) and pcDNA myc tagged AGO-2 was a kind gift from Dr. Greg Hannon at CSHL (Liu et al, 2005).

For C. elegans, lysates were prepared at L4/young adult stage. Briefly, synchronized worms at L4/young adult stage were washed with IP lysis buffer (ThermoFisher Scientific, Waltham, Mass.) with EDTA-free protease inhibitors (Roche, Basel, Switzerland), resuspended in a small volume of the same lysis buffer followed by snap-freezing in a dropwise fashion in liquid nitrogen leading to formation of beads, and then stored at −80° C.

Lysates were prepared by grinding frozen worm beads in liquid nitrogen using a pre-chilled metal mortar and pestle, followed by sonication as described earlier under “C. elegans lysis”. Worm lysates were pre-cleared using a Pierce™ control agarose resin (ThermoFisher Scientific, Waltham, Mass.) followed by an overnight incubation at 4° C. with g of ALG-1 antibody for IP (ThermoFisher Scientific, Waltham, Mass.) using 10 mg of total worm lysates, with rotation. All subsequent washing steps were carried out at 4° C. and elution was carried out room temperature in glycine-HCL buffer, pH 3.5. Immunoblotting was performed using an AIN-1 antibody, which was a kind gift from Dr. John K Kim at Johns Hopkins University.

### Site-Directed Mutagenesis

Site-directed mutagenesis was performed using the QuikChange kit (Agilent Technologies, Santa Clara, Calif.). The sequence of the primers used for mutagenesis is as given in the Key Resources Table.

### Luciferase Assays

0.5×10⁵ cells/well Hela cells were plated in wells of 24 well dishes. They were transiently transfected the next day with 2 μg of either WT-AGO2 or C691S-AGO2, plus 100 ng of either Hmga2 3′UTR-WT luciferase (Luc-wt) or Hmga2 3′UTR-mutant7 luciferase (Luc-m7) control. 100 ng pMax-GFP was also co-transfected to normalize for transfection efficiency. Transfections were carried out using Lipofectamine® 2000 transfection reagent (ThermoFisher Scientific, Waltham, Mass.). Six hours after transfection, the NO donor DETA-NONOate (Cayman Chemicals, Ann Arbor, Mich.) was added at 500 μM. The cells were harvested 24 hours post-transfection, using 1× Passive Lysis Buffer (Promega, Madison, Wis.). Renilla luciferase activity was measuredusing the Renilla Luciferase Assay System (Promega, Madison, Wis.) and a Veritas™ microplate luminometer (Turner BioSystems, Sunnyvale, Calif.) and normalized to GFP readings measured using a BMG FluoStar Galaxy microplate reader. Hmga2 3′UTR WT luciferase (Luc-wt) and Hmga2 3′UTR m7 luciferase (Luc-m7) were gifts from David Bartel (Addgene plasmid #14785 and #14788, respectively)).

siRNA Knockdown Experiments

siRNA knockdown experiments were performed in HEK293 cells that had been plated in 6-well plates and transfected with the indicated plasmids using PolyJet reagent (SignaGen Laboratories, Rockville, Md.), following manufacturer's instructions. The siRNA knockdowns were performed using either the β-arrestin2 specific siRNA (Sigma-Aldrich, St. Louis, Mo.) or non-specific siRNA controls (ThermoScientific, Waltham, Mass.) with Lipofectamine RNAi Max from Invitrogen™ (ThermoFisher Scientific, Waltham, Mass.), following manufacturer's instructions.

### Western Blotting

Lysates containing equal amount of protein were run on pre-cast 4-20% polyacrylamide gels (Bio-Rad, Hercules, Calif.) followed by transfer onto Nitrocellulose blotting membrane (GE Healthcare, UK) using the wet-transfer method. The membranes were then blocked with PBS-T containing 5% milk, followed by overnight incubation with specific antibody in 1×PBS containing 5% milk. After multiple washing with 1×PBS-T, the membrane was incubated for 1 hr with HRP-conjugated secondary from the appropriate species. After multiple washes with 1×PBS-T, the membrane was exposed to SuperSignal® West Femto Maximum Sensitivity Substrate (ThermoScientific, Waltham, Mass.) per manufacturer's instructions, followed by autoradiography.

### 3D Structure of AGO1

The published AGO1-GW182 hook 3D crystal structure (sPDB accession number 5W6V) was downloaded from Protein Data Bank, https://www.rcsb.org/pdb/home/home.do. Open-sourced PyMOL 2.0 was used to locate and highlight the residue and regions of interest.

### Quantification and Statistical Analysis

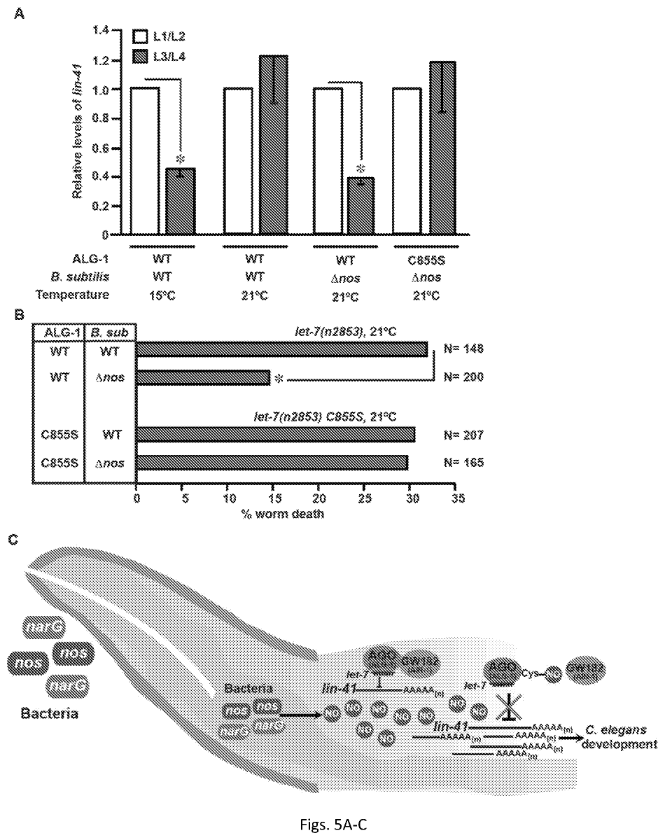

ImageJ software was used for quantification of all Western blot and SDS-PAGE data. Data in Fig.s are represented as mean±SEM. Data were analyzed using KaleidaGraph Software. The p-values were typically calculated as repeated measures ANOVA with Dunnett's post-test statistic unless otherwise stated. In FIG. 5B, vulval bursting was analyzed using the Chi square test for independence after adjusting for multiple comparisons with the Bonferroni correction.

### Results

Microbiota-Derived Nitric Oxide Mediates Protein S-Nitrosylation in C. elegans

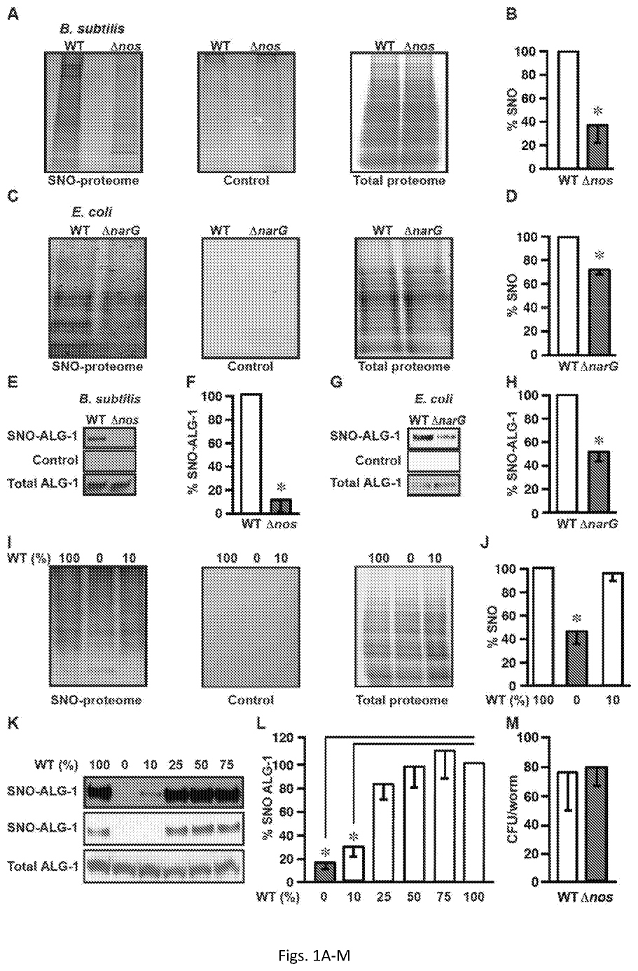

To test the hypothesis that nematode S-nitrosylation is mediated by microbiota-derived NO, we plated microbe-free nematodes (C. elegans, N2 strain) on lawns of either wild-type (WT) B. subtilis, or a mutant strain containing a deletion of the bacterial NOS (Δnos). We then isolated total protein from worms at the L4/young adult stage and specifically pulled down S-nitrosylated proteins using resin-assisted capture (SNO-RAC). We observed large-scale and robust S-nitrosylation of the C. elegans proteome that was dependent on bacterial NOS (FIGS. 1A and 1B). We obtained similar findings in experiments where the nematodes were plated on WT E. coli or mutant E. coli harboring a deletion of nitrate reductase (ΔnarG) (FIGS. 1C and D), which generates NO under anaerobic conditions such as are known to be present in nematode gut. S-nitrosylation of host proteins by dissimilar microbiota under both aerobic and anaerobic conditions suggests that S-nitrosylation may be observed in multiple habitats. Approximately 1000 S-nitrosylated host proteins were identified by mass spectrometry (MS) of worms cultured on WT B. subtilis. KEGG analysis demonstrated an enrichment of proteins involved, for example, in energy utilization and cellular metabolism, recapitulating findings in mammalian cells. The proteome of a metazoan, therefore, can be dramatically altered at the post-translational level by commensal bacteria, in particular via S-nitrosylation.

The widespread modification of the host proteome by its microbiota begs the question of whether these modifications can impact host cellular function(s). The Argonaute-related protein ALG-1 is among the nematode proteins of well-defined function that we identified as being S-nitrosylated in nematodes co-cultured with B. subtilis. The highly conserved ALG-1 protein mediates the post-transcriptional down regulation of mRNAs via the microRNA pathway. As C. elegans has been a classic model for the study of microRNA-dependent gene regulation, including numerous cellular functions, we investigated the possibility that protein modification by resident microbes could regulate host cellular processes via microRNAs. MicroRNA pathways in C. elegans classically regulate the timing of postembryonic cell fate progression and determination across several cell lineages; this regulation is essential to normal development of the animal and ultimately entry into adulthood. As ALG-1 has established functions in worm development, we asked whether microbiota-derived NO might play a role in microRNA-mediated temporal control of gene expression and development. We used an ALG-1 specific antibody to first verify directly that ALG-1 was S-nitrosylated by commensal bacteria. Using SNO-RAC, we demonstrated that ALG-1 was robustly S-nitrosylated in situ and that S-nitrosylation was markedly attenuated in nematodes grown on Δnos B. subtilis (FIGS. 1E and 1F). S-nitrosylation of ALG-1 was also seen in nematodes plated on E. coli and host S-nitrosylation was eliminated with ΔnarG E. coli (FIGS. 1G and H). Thus, S-nitrosylation of C. elegans ALG-1 is mediated by NO derived from the microbiota. That ALG-1 is robustly S-nitrosylated by two different microbes with propensity to generate NO in different amounts and under different conditions, strongly suggests physiological relevance.

To further strengthen the case for physiological relevance, we plated C. elegans on lawns of mixed WT and Δnos B. subtilis, with increasing amounts of WT B. subtilis to determine the minimal percentage of NO-producing bacteria required for detectable interspecies S-nitrosylation. Even a 10% WT B. subtilis mixture was sufficient to achieve protein S-nitrosylation (FIGS. 11 and J) and 25% WT B. subtilis achieved saturating levels of ALG-1 S-nitrosylation, making it highly likely that in native habitats, the C. elegans microbiota produce NO at levels sufficient to mediate interspecies S-nitrosylation (FIGS. 1K and L). In order to test for differences inbacterial abundance within worms plated on WT or Δnos B. subtilis, we quantified bacterial colony formation from homogenized single worms, using methods that allow gut bacteria to remain viable. Supernatant from unlysed worms was used as control (to correct for external contamination). Similar numbers of intact bacteria were found in worms cultured on WT vs. Δnos subtilis (FIG. 1M), consistent with a previous report. Collectively, our results show that nematodes regulate access to NO by varying food intake (amount of bacteria), food source (bacterial species) or oxygen tension in their environment (e.g., depth in soil).

### S-Nitrosylation of Argonaute Proteins at a Phylogenetically-Conserved Cysteine

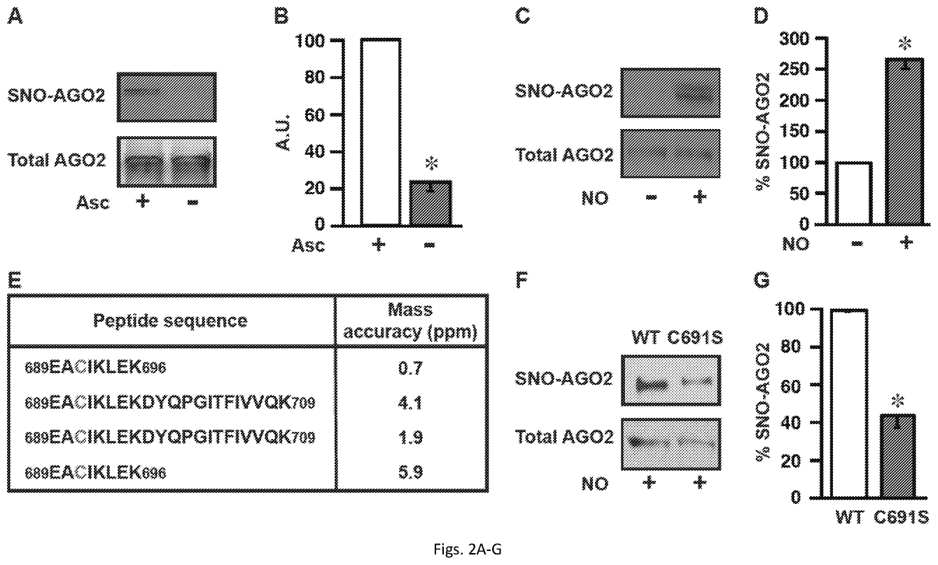

To determine the effect of S-nitrosylation on Argonaute function, we first sought to identify the Cys residue undergoing modification. Since C. elegans can be recalcitrant to biochemical manipulation and because Argonaute proteins are highly conserved, we focused initially on human AGO2 (arguably the primary mammalian Argonaute activity). Notably, we observed that AGO2 was endogenously S-nitrosylated in HEK293 cells (FIGS. 2A, 2B), which express low basal levels of endothelial NOS, and that exogenous NO increased AGO2 S-nitrosylation (FIGS. 2C, 2D). Thus, the molecular machinery of mammalian translational repression is modified by NO (as it is in the nematode) and provides a tractable system for biochemical analysis.

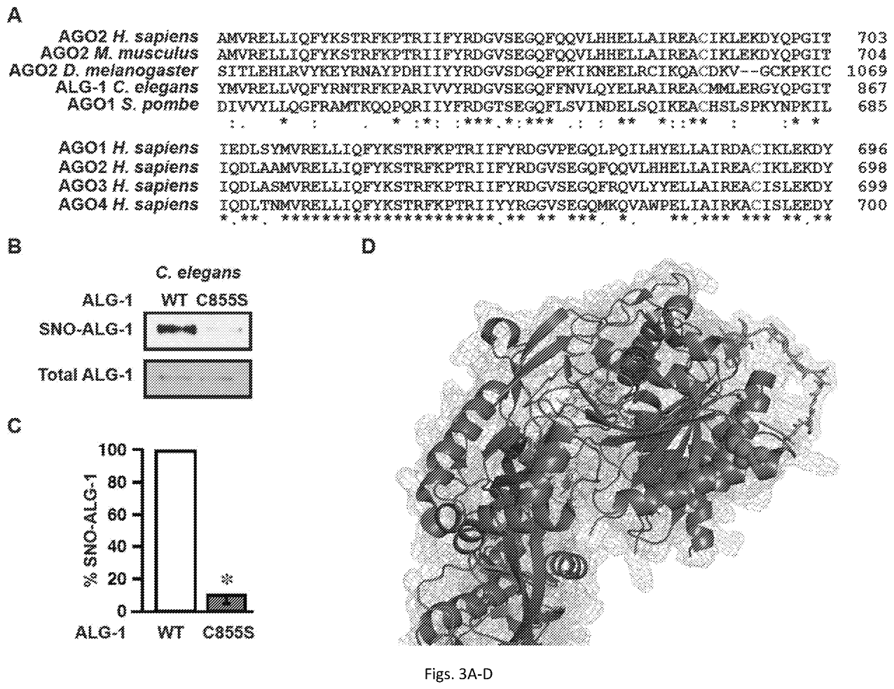

AGO2 has 22 cysteine residues, many of which are predicted by GPS-SNO analysis (a computation algorithm for SNO-site identification) to be putative S-nitrosylation sites. Hence, we undertook an MS-based approach to identify the specific sites of S-nitrosylation. We incubated purified, recombinant human AGO2 with the NO donor S-nitrosocysteine (CysNO; 100 μM), followed by pull-down using an AGO2 antibody. The samples were then subjected to a modified switch assay (Jaffrey and Snyder, 2001), in which NO groups are replaced by iodoacetamide (IAA), and analyzed by LC-MS/MS. Notably, only one cysteine residue, Cys691, was consistently identified as being S-nitrosylated in AGO2 (FIG. 2E). We then confirmed that Cys691 was a primary locus of NO modification by transfecting HEK293 cells with either WT AGO2 or a mutant AGO2 in which Cys691 was replaced by serine (C691S). Upon treatment with CysNO (100 μM), WT AGO2 was strongly S-nitrosylated while the signal was much weaker in the C691S mutant (FIGS. 2F and 2G). Interestingly, Cys691 is highly conserved across phylogeny, including human Argonaute isoforms (AGO1-4) as well as nematode ALG-1 (FIG. 3A). Given the conserved site for S-nitrosylation (Cys855 in ALG-1), we used genome editing to generate a nematode with the C855S point mutation. ALG-1 C855S animals showed markedly lower levels of ALG-1 S-nitrosylation in tissues as compared to their WT counterparts; S-nitrosylation was in fact virtually undetectable in mutant ALG-1 animals (FIGS. 3B and 3C). Thus, Cys855/Cys691 represents a phylogenetically conserved site of S-nitrosylation of Argonaute proteins, and the C855S nematode is essentially refractory to ALG-1 S-nitrosylation.

S-Nitrosylation Inhibits the Essential Interaction of Argonaute-2 with GW182

We next questioned whether S-nitrosylation of AGO2 altered its gene silencing activity. AGO2 is part of a multi-protein assembly that includes GW182 family proteins. The interaction between AGO2 and GW182 is required for silencing of mRNA targets. An inspection of a recent structure of human AGO1 with endogenous RNA and the hook motif of GW182 revealed that the conserved Cys resides within the PIWI domain, adjacent to the putative interaction site with GW182 (FIG. 3D). By contrast, the conserved Cys was distant from the RNA binding pocket, which argues against a role in mediating RNA contacts (FIG. 3D).

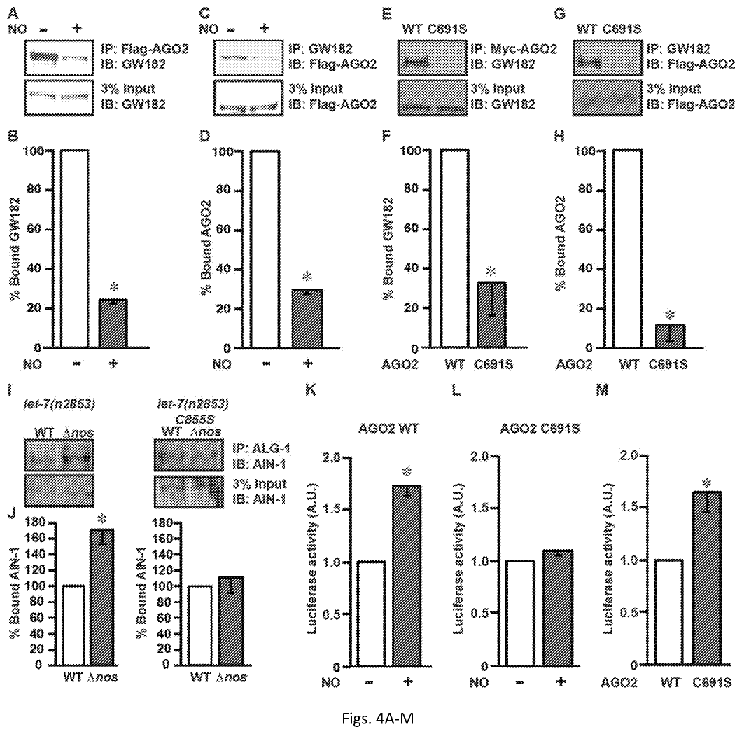

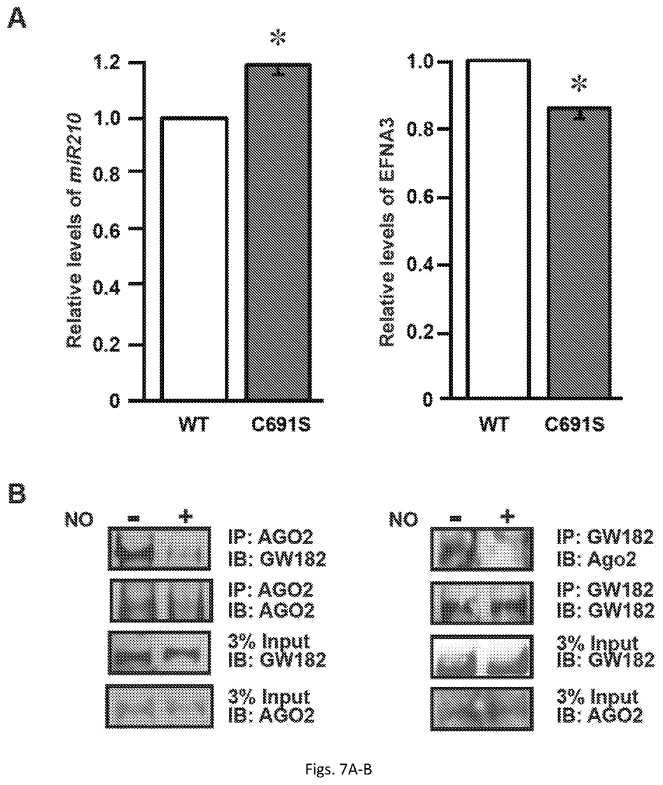

We therefore hypothesized that S-nitrosylation may alter the binding of AGO2 to GW182. In co-immunoprecipitation experiments, WT AGO2 was physically associated with GW182, and this association was strongly inhibited by addition of NO (DETA-NO; see Methods) (FIGS. 4A-4D). Mutation of the S-nitrosylation site to a serine (C691S) markedly decreased the interaction between AGO2 and GW182 (FIGS. 4E-4H), but hardly altered the ability of AGO2 to interact with either microRNA or mRNA (FIG. 7A). This is consistent with other AGO2 mutations that affect its binding to GW182 proteins but do not change microRNA binding. Further, exogenously transfected siRNA, whose activity is independent of GW182 proteins, demonstrated similar knockdown efficiency in HEK293 cells expressing either WT FLAG-AGO2 or C691S FLAG-AGO2. An inhibitory effect of S-nitrosylation on the interaction between endogenous ALG-1 and AIN-1 (the C. elegans GW182 ortholog) was also demonstrated by immunoprecipitations from WT or C855S-ALG-1 worms cultured on either WT or Δnos B. subtilis (FIGS. 41 and 4J). In addition, NO inhibited the interaction between endogenous AGO2 and GW182 in cultured mammalian cells (FIG. 7B). Thus, based on reciprocal co-immunoprecipitations of Argonautes and GW182 proteins in worms and mammals in the presence and absence of NO, all of which show reduced interaction following NO treatment but where this NO effect is also lost after mutation of AGO2-C691S, we conclude that S-nitrosylation of AGO2/ALG-1 inhibits their interaction with GW182 proteins. We also conclude from these data that S-nitrosylation mediated by microbiota may regulate Argonaute/GW182 protein interactions in situ, and that mutation of the Cys site of NO modification (C691 or its C. elegans ortholog at 855) mimics the effect of S-nitrosylation, as it often does in other systems). Thus, Cys691/C855 needs to be in its native (un-nitrosylated) state to interact efficiently with GW182 proteins. Altering this conserved residue by either S-nitrosylation or mutation leads to decreased interaction with GW182, perhaps by disrupting hydrogen bonding interactions or altering the charge distribution at the interface of the two proteins.

S-Nitrosylation of Argonaute Proteins Inhibits miRNA-Mediated Gene Silencing

Our data predict that S-nitrosylation of Cys691 should interfere with AGO2 silencing of mRNA targets. To test this, we used a validated reporter assay where the luciferase gene is flanked by the 3′ UTR of HMGA2 mRNA (a known target of let-7 microRNA) containing either seven WT or seven mutated let-7 binding sites. These reporters were then co-transfected with WT or C691S mutant AGO2, in the absence or presence of NO. Consistent with our hypothesis, WT AGO2 repressed its target poorly in the presence of NO (manifest by higher expression of luciferase mRNA), whereas NO had little effect on mutant C691S AGO2 activity (FIGS. 4K and 4L). Furthermore, mutant C691S AGO2 activity, as measured by luciferase mRNA repression, was weaker than WT AGO2 activity (FIG. 4M) and the relative difference between WT and C691S mutant AGO2 activity was comparable to that of WT AGO2 activity in the absence and presence of NO (FIG. 4M vs. FIG. 4K). Taken together, these findings identify a potential molecular mechanism for microbiota-dependent microRNA-based regulation of gene silencing, whereby exogenous NO mediated S-nitrosylation of a single conserved cysteine in Argonaute proteins disrupts interaction with GW182, and ultimately inhibits miRNA mediated repression of target mRNAs.

Microbial S-Nitrosylation of ALG-1 Influences C. elegans Developmental Timing Via microRNA Activity

To establish a functional role for microbiota-mediated S-nitrosylation, we examined the effect of ALG-1 S-nitrosylation on miRNA-mediated regulation of developmental timing in C. elegans. The let-7 miRNA is conserved between C. elegans and humans, and has been shown to be essential for the advancement of adult cell fate programs in C. elegans. In particular, during vulval morphogenesis, the let-7 miRNA temporally targets lin-41 mRNA upon entry of the animal into the late larval stages; failure to target lin-41 leads to vulval rupture and animal death. Notably, ALG-1-C855S animals (with impaired AIN-1 binding) were difficult to generate and invariably failed to propagate on the WT background, possibly consistent with lethality seen in worms with ALG-1 mutations that disrupt AIN-1 binding.

Let-7 is loaded onto both ALG-1 and ALG-2 proteins, which share developmental functions and targets. Because ALG-1-C855 is also conserved in ALG-2, this redundancy of proteins and potential redundancy of regulation may buffer the effects of NO and protect from developmental defects (predictably, N2 C. elegans fed on either WT or Δnos B. subtilis were similarly capable of let-7 mediated lin-41 silencing at a later larval stage (L3/L4)). Subtler measures of let-7 activity in C. elegans have been assisted by development of sensitizing mutations. We employed the let-7(n2853) temperature sensitive mutant, which fortuitously allowed for C855S propagation. The let-7(n2853) animal is known to experience nearly 100% lethality due to vulval bursting at the non-permissive temperature of 25° C. but none at the permissive temperature of 15° C. While let-7(n2853) mutants at 15° C. demonstrated nearly WT levels of lin-41 repression, mutants incubated at semi-permissive 21° C. were incapable of let-7 mediated lin-41 repression at late larval stages (FIG. 5A). Notably, feeding with Δnos B. subtilis at 21° C. fully rescued the developmental stage-specific lin-41 repression. Moreover, in the C855S mutant lacking the ALG-1 S-nitrosylation site, the rescue of lin-41 repression mediated by eliminating microbe-derived NO was abolished (FIG. 5A). These results strongly suggest that gene repression during development is regulated by microbiota-mediated S-nitrosylation of ALG-1.

We reasoned that lethal vulval rupture of let-7(n2853) mutants, secondary to perturbations in late larval stage-specific miRNA repression of gene expression, namely lin-41, would represent a consistent and quantifiable functional readout of the effect of the microbiota on host development. These mutants held at non-permissive temperatures (25° C.) exhibit lethality consequent to vulval bursting during the larval-to-adult transition, as well as severe gonadal defects. We scored vulval bursting at the intermediate temperature of 21° C., at which these sensitized animals experienced a ˜30% bursting rate when cultured with WT B. subtilis. Remarkably, when incubated with Δnos B. subtilis, the bursting rate was 15%, a full two-fold reduction (p=0.003) (FIG. 5B). This protective effect was independent of levels of mature let-7 miRNA in late larval stages, which were similar in both groups (FIG. S4C). Additionally, no such difference in lethality was observed in C855S let-7(n2853) animals cultured either on WT or Δnos B. subtilis (FIG. 5B), and C855S let-7(n2853) mutants displayed lethality that was virtually identical to let-7 (n2853) animals (i.e., WT ALG-1) cultured on WT B. subtilis. Thus our data indicate that commensal bacteria directly modify C. elegans ALG-1 via S-nitrosylation to alter host gene expression and thereby impact host developmental timing and phenotypic outcome (FIG. 5C).

We have discovered that the host microRNA machinery is regulated by microbial NO through a locus conserved among Argonaute proteins. Interspecies S-nitrosylation thereby regulates host gene expression via microRNAs, opening new avenues of investigation. Further, we describe the functional consequences of microbiota-control of animal physiology in regulation of vulval development.

There is growing appreciation of the multiple sources of NO that may influence animal fitness, including NOSs, cytochrome c oxidase, nitrate reductases and nitrate-rich foods. Irrespective of its source, NO is converted in situ into bioactive S-nitrosothiols that convey NO bioactivity and mediate S-nitrosylation of proteins. In this model, dedicated signal transduction pathways, not the source of NO, determine cellular responses. This can be best appreciated in the blood pressure-lowering effect of NO generated from gastric byproducts, despite the ubiquitous presence of NOS throughout the circulatory system. S-nitrosothiols in the stomach can evidently access vascular tissues throughout the body to regulate end-organ effects independently of NO produced locally. Pathways that convey self-versus nonself-derived NO bioactivity and accordingly partition cellular signals into uniquely tailored responses remain tobe elucidated. This is a matter of importance as microbial NO contributes far more to the host nitrosoproteome than previously imagined.

C. elegans represents a notable example of an animal where NO is not derived primarily from NOS. While alternative sources of endogenous NO evidently exist (FIG. 1), C. elegans' reliance on commensal bacteria has allowed for investigation, at unprecedented molecular and mechanistic detail, into microbiota-regulated host signaling-from the source of the bioactive signaling molecule in B. subtilis and E. coli to its function-regulating modifications of host proteins. The potential implications of these data for mammalian biology are tantalizing, as the mouth and skin microbiota in humans are known to represent functional sources of NO that can affect cardiovascular homeostasis and host energy utilization. In addition, gut-derived NO has been shown to S-nitrosylate plasma albumin, establishing the principle that NO derived from food and enteric origins can promote S-nitrosylation of host proteins. Given the potential generality of our findings, including the conservation of Argonaute S-nitrosylation sites and of S-nitrosylation sites generally, it is reasonable that human microbiome (and food)-derived NO influences gene regulation via miRNAs and host cellular functions broadly.

Post-translational modifications are determinants of Argonaute protein function. As central effectors of the miRNA pathway, Argonaute proteins are increasingly recognized as subject to diverse modifications. For example, Argonaute protein stability is regulated by hydroxylation of proline residues, while S387 phosphorylation facilitates RISC formation by increasing AGO2 interaction with cofactors like GW182 to enhance gene repression. Our data represent the first report of S-nitrosylation of Argonaute proteins in regulating the miRNA pathway. By contrast to serine phosphorylation, we find that S-nitrosylation of AGO2 serves to destabilize its association with GW182 leading to reduced miRNA activity. The site of S-nitrosylation in AGO2 is conserved in C. elegans ALG-1, preventing its association with the GW182 ortholog AIN-1 in situ. Thus, microbial regulation of the host miRNA machinery is a physiological occurrence in living animals. Taken together, our findings show that S-nitrosylation of Argonaute proteins transduces both microbiota- and host-derived signals to regulate gene expression.

The striking influence of the microbiota on organ developmental timing (through modulation of lin-41 activity; FIG. 5) may reflect host-microbe co-evolution. Studies in human twin pairs reveal the impact of host genetics on the microbiome. Although the fidelity of any specific microbial partner-host relationship is highly variable, C. elegans isolated from their native habitats retain a core bacterial community with several NO producing species; hence host responsiveness to bacterially-derived NO may have conferred a survival benefit, perhaps through effects on development. B. subtilis, which we study, is found among the natural microbiota of C. elegans and although E. coli itself is not, other bacteria that, like E. coli, use nitrate reductase to generate NO are found naturally in the worm microbiome, such as members of the phylum Actinobacteria. Whether C. elegans seek out such bacteria or are colonized by NO-producing microbes, and whether they may regulate their own exposure to NO (e.g., by varying the amount or species of bacteria they consume or the oxygen tension in their environment through their depth in soil) remains to be seen.

The broad spectrum of proteins we observed in the nematode nitrosoproteome (of which we list ˜1000 using screening methodology that provides only a partial picture) implies that wide-ranging host physiology, beyond miRNA control of developmental timing, may be regulated by commensal bacteria. As one example, it has been reported that the increase in C. elegans longevity mediated by commensal NO requires the mammalian FOXO orthologue DAF-16, although the molecular mechanism is not known. We find that DAF-16 is robustly S-nitrosylated by microbial NO under standard laboratory conditions (growth on E. coli), suggesting that the effects of bacterial NO on lifespan may be mediated by transcriptional regulation. Further, a recent study has observed that P. aeruginosa-generated NO contributes to avoidance behavior that is dependent on the SNO-regulating activity of C. elegans protein thioredoxin-1 (TRX-1), although the SNO-targets have yet to be identified. Notably, we find thioredoxin-like 1 (TXL-1) and thioredoxin reductase (TRXR-1) in our C. elegans SNO-proteome. More generally, it may be fruitful to consider whether signaling by gasotransmitters as a rule (including NO, H₂S and CO) represents a general strategy for interspecies communication involving modification of host proteomes. Thus, microbiota-dependent modification of host proteomes by gasotransmitters-exemplified by robust interspecies S-nitrosylation-represents a general mechanism by which the resident microbiota control host functions. We also reason that intake of dietary sources of NO in mammals might have physiologic consequences during early developmental stages.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of treating a disorder associated gasotransmitter signaling in a subject in need thereof, the method comprising,
modulating the microbiota of the subject to modulate microbiota gasotransmitter production and modification of the subject's proteome to treat the disorder in the subject.

2. The method of claim 1, wherein the gasotransmitters comprise at least one of NO, H₂S, or CO.

3. The method of claim 1, wherein the microbiota of the subject's gastrointestinal tract is modulated to modulate microbiota gasotransmitter production.

4. The method of claim 1, wherein the microbiota of at least one of the subject's stomach and intestines are modulated to modulate microbiota gasotransmitter production.

5. The method of claim 1, wherein the microbiota of the subject's oral cavity is modulated to modulate microbiota gasotransmitter production.

6. The method of claim 1, wherein the subject has a dysbiosis of the gastrointestinal microbiota.

7. The method of claim 1, wherein the gasotransmitter comprises NO and the disorder is associated with NO/SNO deficiency or those benefiting from increased SNO in the subject.

8. The method of claim 7, wherein the microbiota is modulated by administering to the gastrointestinal tract bacteria and/or fungi capable of producing nitric oxide and optionally a substrate of nitric oxide synthase and/or nitrate reductase.

9. The method of claim 8, wherein the bacteria is genetically modified to express or over express nitric oxide synthase or nitrate reductase.

10. The method of claim 8 wherein the substrate of nitric oxide synthase or nitrate reductase comprises at least one of arginine, a nitrate, a nitrite, of a salt thereof,

11. The method of claim 10, wherein the nitrate is a botanical source of nitrate and the botanical source of nitrate comprises one or more of beet root, kale, artichoke, holy basil, gymnema sylvestre, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof.

12. The method of claim 8, wherein the bacteria and the substrate of nitric oxide synthase and/or nitrate reductase are administered at the same time.

13. The method of claim 8, wherein the bacteria comprises commensal bacteria.

14. The method of claim 13, wherein the commensal bacteria comprise at least one bacteria of the genera Alistipes, Akkermansia, Anaerofilum, Bacteroides, Blautia, Bifidobacterium, Butyrivibrio, Clostridium, Coprococcus, Dialister, Dorea, Fusobacterium, Eubacterium, Faecalibacterium, Lachnospira, Lactobacillus, Odoribacter, Oscillospira, Parabacteroides, Phascolarctobacterium, Peptococcus, Peptostreptococcus, Prevotella, Roseburia, Ruminococcus, Streptococcus, or Subdoligranulum.

15. The method of claim 8, wherein the bacteria/fungi and optionally the substrate are administered to the subject at an amount effective to increase S-nitrosylation of proteins in the subject.

16. The method of claim 8, wherein the bacteria/fungi and optionally the substrate are administered to the subject at an amount effective to increase SNO levels in blood or tissue of the subject.

17. The method of claim 8, wherein the disorder comprises ischemia.

18. The method of claim 17, wherein the ischemia comprises ischemic tissue or tissue damaged by ischemia.

19. The method of claim 8, wherein the bacteria and optionally the substrate are administered to the subject at an amount effective to treat at least one of acute coronary syndrome, acute lung injury (ALI), acute myocardial infarction (AMI), acute respiratory distress syndrome (ARDS), pulmonary fibrosis, asthma, COPD, arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasia, ischemic vascular disease, hyperoxic injury, hypoxia, inflammation, inflammatory bowel disease, , irritable bowel syndrome, constipation, anal fissures, anal spasm, duodenal and gastric ulcers, pyloric stenosis, sphincter of oddi constriction, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, neurodegenerative disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, or wounds to tissues or organs.

20. A method of treating a disorder associated with NO/SNO deficiency or those benefiting from decreased SNO in a subject in need thereof, the method comprising, administering to the gastrointestinal tract bacteria (and/or fungi) capable of producing nitric oxide and optionally a substrate of nitric oxide synthase and/or nitrate reductase to treat the disorder in the subject.

21. The method of claim 20, wherein the bacteria and optionally the substrate are administered to the subject's stomach and intestines.

22. The method of claim 20, wherein the bacteria is genetically modified to express or over express nitric oxide synthase or nitrate reductase.

23. The method of claim 20, wherein the substrate of nitric oxide synthase or nitrate reductase comprises at least one of arginine, nitrate, nitrite, of a salt thereof.

24. The method of claim 23, wherein the nitrate is a botanical source of nitrate and the botanical source of nitrate comprises one or more of beet root, kale, artichoke, holy basil, gymnema sylvestre, ashwagandha root, salvia, St. John wort, broccoli, stevia, spinach, gingko, kelp, tribulus, eleuthero, epimedium, eucommia, hawthorn berry, rhodiola, green tea, codonopsys, panax ginseng, astragalus, pine bark, dodder seed, Schisandra, cordyceps, and mixtures thereof.

25. The method of claim 20, wherein the bacteria and the substrate of nitric oxide synthase and/or nitrate reductase are administered at the same time.

26. The method of claim 20, wherein the bacteria comprise commensal bacteria.

27. The method of claim 26, wherein the commensal bacteria comprise at least one bacteria of the genera Alistipes, Akkermansia, Anaerofilum, Bacteroides, Blautia, Bifidobacterium, Butyrivibrio, Clostridium, Coprococcus, Dialister, Dorea, Fusobacterium, Eubacterium, Faecalibacterium, Lachnospira, Lactobacillus, Odoribacter, Oscillospira, Parabacteroides, Phascolarctobacterium, Peptococcus, Peptostreptococcus, Prevotella, Roseburia, Ruminococcus, Streptococcus, or Subdoligranulum.

28. The method of claim 20, wherein the bacteria and optionally the substrate are administered to the subject at an amount effective to increase S-nitrosylation of proteins in the subject.

29. The method of claim 20, wherein the bacteria and optionally the substrate are administered to the subject at an amount effective to increase SNO levels in blood or tissue of the subject.

30. The method of claim 20, wherein the disorder comprises ischemia.

31. The method of claim 30, wherein the ischemia comprises ischemic tissue or tissue damaged by ischemia.

32. The method of claim 20, wherein the bacteria and optionally the substrate are administered to the subject at an amount effective to treat at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasia, ischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, or wounds to tissues or organs.

33. A method of treating a disorder associated a change in gene expression in a subject in need thereof, the method comprising,
modulating the microbiota of the subject to modulate microbiota gasotransmitter production and modification of the subject's proteome to treat the disorder in the subject.

34. A method of treating a disorder associated with change in microRNA activity in a subject in need thereof, the method comprising,
modulating the microbiota of the subject to modulate microbiota gasotransmitter production and modification of the subject's proteome to treat the disorder in the subject.

35. A method of treating a disorder associated gasotransmitter signaling in a subject in need thereof, the method comprising,
modulating the microbiota of the subject to modulate microbiota gasotransmitter production and modification of the subject's proteome to treat the disorder in the subject.

36. The method of claim 35, wherein the microbiota and/or subject are administered inhibitors of gasotransmitter production by the microbiota at an amount effective to decrease S-nitrosylation of proteins in the subject.

37. The method of claim 35, wherein the microbiota and/or subject are modulated to increase S-nitrosylation of proteins in the subject.

38. The method of claim 37, wherein the microbiota and/or subject are modulated to increase S-nitrosylation of cellular proteins in the subject.

39. The method of claim 37, wherein the microbiota and/or subject are modulated to increase S-nitrosylation of blood proteins in the subject.

40. The method of claim 35, wherein the microbiota and/or subject are administered inhibitors of gasotransmitter production by microbiota at an amount effective to decrease S-sulfhydration of proteins in the subject.

41. The method of claim 35, wherein the microbiota and/or subject are administered inhibitors of gasotransmitter production by microbiota at an amount effective to increase S-oxidation of proteins in the subject.

42. The method of claim 35, wherein the microbiota and/or subject are modulated to increase S-sulfhydration of proteins in the subject.

43. The method of claim 35, wherein the microbiota and/or subject are administered inhibitors of gasotransmitter production by microbiome at an amount effective to decrease CO microbiota production in the subject.

44. The method of claim 35, wherein the microbiota and/or subject are modulated to increase CO microbiota production in the subject.
